(19) United States
(12) Patent Application Publication
(10) Pub. No.: US 2025/0241901 A1
(43) Pub. Date: Jul. 31, 2025

(54) ACTIVATION OF MNRR1 / CHCHD2 AS A THERAPEUTIC TARGET FOR MITOCHONDRIA ASSOCIATED DISORDERS

(71) Applicant: Wayne State University, Detroit, MI (US)

(72) Inventors: Lawrence I. Grossman, Detroit, MI (US); Siddhesh Aras, Detroit, MI (US)

(73) Assignee: Wayne State University, Detroit, MI (US)

(21) Appl. No.: 19/043,397

(22) Filed: Jan. 31, 2025

Related U.S. Application Data
(60) Provisional application No. 63/627,551, filed on Jan. 31, 2024.

(51) Int. Cl. A61K 31/426; A61K 9/00; A61K 31/57; A61P 15/06; A61P 29/00

(52) U.S. Cl. CPC A61K 31/426; A61K 9/0053; A61K 31/57; A61P 15/06; A61P 29/00

(57) ABSTRACT

The present disclosure relates generally to compositions and methods for treating inflammatory disorders or mitochondria-associated disorders by activation of Mitochondrial Nuclear Retrograde, Regulator 1 (MNRR1 or CHCHD2).

### CROSS-REFERENCE TO RELATED APPLICATION

This application claims priority to and the benefit of the earlier filing date of U.S. Provisional Application No. 63/627,551, filed on Jan. 31, 2025, which is incorporated by reference herein in its entirety.

### STATEMENT REGARDING FEDERALLY SPONSORED RESEARCH OR DEVELOPMENT

This invention was made with government support under contracts W81XWH-21-1-0402 and W81XWH-16-1-0516 awarded by the Department of Defense. The government has certain rights in the invention.

### INCORPORATION BY REFERENCE OF SEQUENCE LISTING

A computer readable text file, entitled “W063-6001 US_SeqList.xml” created on or about Jan. 31, 2025, with a file size of 28,672 bytes, contains the sequence listing for this application and is hereby incorporated by reference in its entirety.

### FIELD OF THE DISCLOSURE

The present disclosure relates generally to activating Mitochondrial Nuclear Retrograde, Regulator 1 (MNRR1/CHCHD2) as therapy for inflammatory disorders and mitochondria-associated disorders. Nitazoxanide is demonstrated as an effective activator of MNRR1, including in preventing inflammation-induced preterm birth.

### BACKGROUND OF THE DISCLOSURE

Intra-amniotic inflammation leading to preterm birth is one of the primary causes of neonatal morbidity and mortality (Romero et al., Semin Fetal Neonatal Med, 11:317-326, 2006; Yoon et al., Am. J. Obstet. Gynecol., 182:675-681, 2000; Villamor-Martinez et al., JAMA Netw. Open, 2: e1914611, 2019). Intra-amniotic inflammation results from ascending microbial invasion from the lower genital tract to the amniotic cavity, termed intra-amniotic infection, or from endogenous danger signals (i.e., alarmins) released upon cellular damage or stress, known as sterile intra-amniotic inflammation (Villamor-Martinez et al., JAMA Netw. Open, 2: e1914611, 2019; Romero et al., Am. J. Reprod. Immunol., 72:458-474,2014; Romero et al., J. Matern. Fetal Neonatal Med., 28:1394-1409, 2015). Although immune cells are known to play a key role in the response to inflammation at the fetal-maternal interface (Miller et al., J. Immunol., 209:1450-1464, 2022; Gomez-Lopez et al., Cell. Mol. Immunol., 11:571-581, 2014), the specific mechanisms underlying the response of cells in the placental tissues to inflammatory insults are largely unknown (Cobo et al., Am. J. Obstet. Gynecol., 211:708, 2014).

Recently, a non-canonical, TLR4-independent signaling pathway involving mitochondria in placental cells that contributes to immune pathology was identified (Purandare et al., iScience, 25: 105342, 2022; Purandare et al., iScience bioRxiv Nov. 7, 2021, doi.org/10.1101/2021.11.06.467519). Using in vitro and in vivo models, the mitochondrial levels of MNRR1 (Mitochondrial Nuclear Retrograde, Regulator 1; also called CHCHD2, AAG10, or PARK22), an important biorganellar regulator of cellular function (Aras et al., Nucleic Acids Res., 41:2255-2266, 2013; Aras et al., Mitochondrion, 20:43-51, 2015; Purandare et al., J. Biol. Chem., 293:6517-6529, 2018; Aras et al., Biochim. Biophys. Acta Mol. Cell Res., 1864:440-448, 2017; Grossman et al., Oxid. Med. Cell. Longev., 2017: 6739236, 2017), were found to be reduced in the context of inflammation. However, whether MNRR1 is implicated in the pathophysiology of intra-amniotic inflammation-induced preterm labor and birth had not been well explored. See also Sato et al. (Hum Mol Genet. 30:443-453, 2021), and Liu et al. (Hum Mol Genet, 29(9):1547-1567, 2020).

MNRR1 functions in two cellular compartments: the mitochondrion and the nucleus. Mitochondrial MNRR1 binds to cytochrome c oxidase (Aras et al., Mitochondrion, 20:43-51, 2015) or Bcl-xL (Liu et al., Cell Death Differ., 22:1035-1046, 2015) to regulate the mitochondrial roles of energy generation and apoptosis, respectively. Nuclear MNRR1 can function as a transcriptional regulator to modulate the activation of stress-responsive genes including MNRR1 itself (Aras et al., Mitochondrion, 20:43-51, 2015; Aras et al., Proc. Natl. Acad. Sci. U.S.A, 117:32056-32065, 2020). This LPS-induced inflammatory signaling pathway was characterized in vitro by using a human trophoblast cell line (HTR8/SVneo) (Purandare et al., iScience, 25: 105342, 2022). The pro-inflammatory pathway is initiated by activation of NOX2, generating radical oxygen species (ROS) that activate ATM kinase, which then phosphorylates and thereby stabilizes YME1L1, a mitochondrial intermembrane space protease that subsequently reduces mitochondrial MNRR1 levels. Furthermore, overexpression of MNRR1 was shown to dampen this inflammatory signaling pathway and to restore mitochondrial function.

### SUMMARY OF THE DISCLOSURE

Nitazoxanide is currently used as a treatment for several infectious organisms including protozoa, helminths, bacteria, and viruses; it clinically approved for the treatment of diarrhea caused by Giardia lamblia or Cryptosporidium parvum. The molecular target of nitazoxanide is known in protozoa, but no target has been identified in humans.

It is demonstrated herein that nitazoxanide can abrogate preterm birth in an LPS-based mouse model of intra-amniotic inflammation by stimulating the transcription of MNRR1. The anti-inflammatory effects of nitazoxanide in pregnant mice are mediated through transcriptional activation of MNRR1, since there is no nitazoxanide-mediated benefit in MNRR1 knockout cells. There was also a basal increase in TNF transcription after MNRR1 knockout that was restored to control levels upon reintroduction of MNRR1.

Inflammation-induced preterm birth leads to significant adverse effects on maternal and fetal health. Administration of nitazoxanide in pregnant mice injected with LPS prevented preterm birth and reduced neonatal mortality up to 3 weeks. This treatment also significantly reversed the inflammation induced by LPS in these animals. Hence, this clinically approved drug is demonstrated herein as a candidate for potential use in high-risk pregnancies (including human pregnancies) that may result in preterm birth. Byway of specific example, nitazoxanide is believed to be useful as a therapeutic strategy for sterile intra-amniotic inflammation, which condition currently lacks treatment.

More generally, with the disclosure herein of successful treatment using nitazoxanide to increase MNRR1 activity and therefore reduce or reverse inflammation in a preterm birth model, also enabled are other methods of treating or reversing other diseases or conditions that involve or are mediated by inflammation.

The current disclosure provides methods of delaying inflammation-induced labor in a pregnant subject, which methods involve selecting a pregnant subject, and activating MNRR1 in a cell of the pregnant subject. By way of example, the cell is a placental cell.

In examples of these methods, wherein activating MNRR1 in the cell of the pregnant subject includes administering an effective amount of nitazoxanide to the pregnant subject. Optionally, the nitazoxanide is administered prior to exposure of the pregnant subject to an inflammation-causing agent (such as a microbe or a chemical or environmental agent).

In examples of the provided method embodiments, delaying inflammation-induced labor in the pregnant subject prevents premature birth.

In example method embodiments, activating MNRR1 in the cell of the pregnant subject includes administering an effective amount of a MNRR1 activating agent to the pregnant subject. By way of example, the MNRR1 activating agent may include at least one active tizoxanide moiety. One specific example MNRR1 activating agent is nitazoxanide. In any of the provided method embodiments, the nitazoxanide is administered to the pregnant subject in a dose of 500 mg, for instance twice daily.

Another provided embodiment is a method of reducing or preventing pre-term labor in a pregnant subject, the method including: selecting a pregnant subject; and administering a MNRR1 activating agent to the pregnant subject. In examples of this embodiment, the MNRR1 activating agent includes at least one active tizoxanide moiety. One exemplary MNRR1 activating agent so nitazoxanide.

In any of the provided method embodiments, it is contemplated that the MNRR1 activating agent can be administered prior to exposure of the pregnant subject to an inflammation-causing agent. An example inflammation-causing agent includes a microbe.

In any of the provided method embodiments, the administering may be oral administration.

In any of the provided method embodiments, reducing or preventing pre-term labor in the pregnant subject prevents premature birth.

Any of the provided method embodiments will optionally include administering an effective amount of progesterone to the pregnant subject—for instance as a concurrent of sequential combination therapy.

Yet another embodiment is a method of treating an inflammatory disorder or a mitochondria-associated disorder in a subject, including activating Mitochondrial Nuclear Retrograde, Regulator 1 (MNRR1) in a cell of the subject.

### BRIEF DESCRIPTION OF THE DRAWINGS

Some of the drawings submitted herein may be better understood in color. Applicant considers the color versions of the drawings as part of the original submission and reserve the right to present color images of the drawings in later proceedings.

FIGS. 1A-1D. Nitazoxanide's anti-inflammatory effects are via transcriptional activation of MNRR1. (FIG. 1A) Results of a screen of ˜2400 FDA-approved drugs and natural compounds identified to transcriptionally activate (>1), inhibit (<1), or not affect (=1) MNRR1. Each circle represents one drug and the MNRR1 activator (nitazoxanide (N)) has been highlighted in green. (FIG. 1B) Equal numbers of HTR cells overexpressing the MNRR1-luciferase reporter were plated on a 96 well plate and treated with increasing amounts of nitazoxanide or vehicle (DMSO) for 24 h. EC₅₀ was calculated using the activation of MNRR1 reporter relative to vehicle treated cells using the Quest Graph^{T}M EC₅₀ Calculator. (FIG. 1C) TNF transcript levels were measured in human placental cells. Actin was used as a housekeeping gene for normalization. Abbreviations: WT, MNRR1-WT; MNRR1-KO, knockout of MNRR1; C, control (water); LPS, lipopolysaccharide; N, nitazoxanide; KO+WT-MNRR1, knockout cells +MNRR1−WT overexpression. n=4 biological replicates; significance: *, p-value<0.05; ns is non-significant. (FIG. 1D) Cell culture supernatants from control (water) or LPS-treated cells were tested as described in Materials and Methods for multiple cytokines using a membrane array. The sample in the bottom right corner is a blank negative control and the remaining three corner samples are proprietary positive controls.

FIGS. 2A-2F. Nitazoxanide prevents preterm delivery and improves neonatal mortality in vivo. (FIG. 2A) Schematic illustration depicting the administration of nitazoxanide (marketed under the commercial name Alinia) and LPS in the murine model of preterm birth. (FIG. 2B) Gestational length of mice was obtained using video-monitoring until delivery (n=6-8 animals). (FIG. 2C) Neonatal mortality was determined by calculating the number of dead pups over total litter size (n=6-8 animals). (FIG. 2D) Survival rates of neonates born to dams intra-amniotically injected with LPS (line) or intra-amniotically injected with LPS and treated with nitazoxanide (line with X) up to 3 weeks of age are displayed as Kaplan-Meier survival curves. No pups from LPS-injected dams survived beyond week 0 of birth. The statistical comparison for week 0 is shown in FIG. 1C. (FIG. 2E) MNRR1 transcript levels were measured in mouse decidual tissue (17.5 dpc). Actin was used as a housekeeping gene to normalize expression (n=4 animals for all except PBS+N, where n=3). (FIG. 2F) Equal amounts of whole placental lysates were separated on an SDS-PAGE gel and probed for MNRR1 levels. Actin was used as a loading control. Gel is representative of data obtained from 2 animals. Keyfor FIGS. 2B, 2C, and 2E: PBS, phosphate buffered saline injection; LPS, lipopolysaccharide injection; PBS+N, PBS injection with oral gavage of nitazoxanide; LPS+N, LPS injection with oral gavage of nitazoxanide. Significance: *, p<0.05; **, p<0.01.

FIGS. 3A & 3B. The transcript levels of several inflammatory factors such as 116, Illb, Tnf, and Tlr4, among others, were assessed in the decidual samples and increased expression was found in dams intra-amniotically injected with LPS that was abrogated upon treatment with nitazoxanide. These results are illustrated with a heatmap (FIG. 3A) and bar graphs (FIG. 3B).

FIGS. 4A-4B: High throughput screen to identify MNRR1 activators. FIG. 4A: Campaign view representing compounds that activate MNRR1. Control levels are represented as a dotted line. Each compounds is shown as a small dot and nitazoxanide is highlighted as a large dot. FIG. 4B: MDAMB468 cells treated with Vehicle (DMSO) or nitazoxanide (10 μM) for 24 h were immunostained (MNRR1=orange, DAPI=blue) and imaged at 63λ using a confocal microscope. The scale bar represents 200 μm.

FIGS. 5A-5E: FIG. 5A: Venn diagram showing activators identified in HEK293 and MDA-MB-468 cells. FIG. 5B: Equal amounts of MELAS cells treated with Vehicle (DMSO) or various MNRR1 activating compounds (10 μM) for 24 h were separated on an SDS-PAGE gel and probed for MNRR1 levels. Actin was probed as a loading control and numbers below represent an average and standard deviation (SD) of two biological replicates. FIGS. 5C-5E: Equal amounts of cell lysates from various cell lines treated with vehicle (DMSO) or tizoxanide (10 μM) for 24 h were separated on an SDS-PAGE gel and probed for MNRR1 plus loading controls GAPDH or tubulin.

FIGS. 6A-6C: FIG. 6A: Above, Chemical structures of nitazoxanide, its metabolites, and similar compounds. Abbreviations: P4, Phenyl-4-aminosalicylic acid; 4ABA, 4-Aminobenzanilide; 4ASA, 4-Amino salicylic acid; BA, Benzanilide; PBO, Phenyl benzoate; PSA, Phenyl salicylic acid; 2AN, 2-Amino 5-nitrothiazole. Below, Equal amounts of lysates of MELAS cells treated with Vehicle (DMSO) or the various compounds (10 μM) for 24 h were separated on an SDS-PAGE gel and probed for MNRR1 levels. Actin was probed as a loading control and numbers below represent an average and SD of 3 biological replicates. FIGS. 6B-6C: MNRR1 transcript levels are shown relative to 18S rRNA (n=4 biological replicates, error bars represent SE). In all figures, * indicates p<0.05, ** indicates p<0.005.

FIGS. 7A-7E: MNRR1 activation by tizoxanide enhances mtDNA biogenesis and mitophagy to shift heteroplasmy in MELAS cells. FIG. 7A: Oxygen consumption measured from MELAS cybrid cells treated with vehicle (DMSO) or tizoxanide (10 μM) for 48 h (n=4 biological replicates, error bars represent SE). FIG. 7B: Haelll restriction enzyme digestion of a PCR-amplified fragment of mtDNA harboring the m.3243A>G mutation from DW7 cells treated with vehicle (DMSO) or tizoxanide (10 μM) for 144 h. The table shows n=4 biological replicates as mean±SD. FIG. 7C: Genes identified using qPCR of MELAS cybrid cells stably overexpressing EV or MNRR1. FIG. 7D: MtDNA levels are shown relative to nuclear DNA (nDNA) (GAPDH) (n=5 biological replicates, error bars represent SE). FIG. 7E: Equal amounts of MELAS cybrid cell lysates, treated with vehicle (DMSO) or tizoxanide (10 μM) for 48 h, were separated on an SDS-PAGE gel and probed for PGC1a, PINK1, LC3 A/B, MNRR1, and Tubulin. FIG. 7F: Equal numbers of MELAS cells were treated as in FIG. 7E, separated on an SDS-PAGE gel, and probed for phospho-ubiquitin (S65) levels. Tubulin was probed as a loading control. In all figures, * indicates p<0.05, ** indicates p<0.005.

FIGS. 8A-8D: MNRR1 activation using tizoxanide enhances mitochondrial function, mitophagy, and protects from LPS-induced inflammation in MELAS patient fibroblasts. FIG. 8A: Equal amounts of lysates from MELAS patient fibroblast (MF 1, 2, or 3) lysates treated with vehicle (DMSO) or tizoxanide (10 μM) for 48 h were separated on an SDS-PAGE gel and probed for LC3B, PGC1a, MNRR1, MTCO2, TOM20, and phospho-ubiquitin (S65) levels. GAPDH orTubulin was probed as a loading control. FIG. 8B: Oxygen consumption measured from MELAS patient fibroblasts treated with vehicle (DMSO) or tizoxanide (10 μM) for 48 h (n=4 biological replicates, mean±SE). FIG. 8C: Mitochondrial ATP rate measured in MELAS patient fibroblasts treated with vehicle (DMSO) or tizoxanide (10 μM) for 48 h (n=4 biological replicates, mean±SE). FIG. 8D: Heatmap representation of ROS and TNFα transcript levels from MELAS patient fibroblasts treated with vehicle (DMSO), LPS (DMSO+500 ng/mL LPS), or LPS plus tizoxanide (10 μM tizoxanide+500 ng/mL LPS) for 24 h (n=4 biological replicates). In all figures, * indicates p<0.05, ** indicates p<0.005.

FIGS. 9A-9E: Nitazoxanide acts by reducing HIF2α levels in MELAS cells. FIG. 9A: Schematic representation of deleted regions of the MNRR1 promoter. FIG. 9B: Dual luciferase reporter assay using the MNRR1 promoter deletions in cells treated with vehicle (DMSO), nitazoxanide, or tizoxanide (10 μM) for 24 h (n=4 biological replicates). FIG. 9C: DNA sequence of region of 800-952 bp in the MNRR1 promoter (SEQ ID NO: 25) highlighting binding sites of the transcription factors shown. FIG. 9D: Transcript levels of HIF2α and HIF1α in MELAS cybrid cells harboring different levels of heteroplasmy (0% to 100%). Data from [6]. FIG. 9E: Protein levels of HIF2α and HIF1α in cybrid cells with 0% (CL9) and 70% MELAS heteroplasmy (DW7) In all figures, * indicates p<0.05, ** indicates p<0.005.

FIGS. 10A-10E: The reduction of MNRR1 in MELAS cells is via HIF2α acting at the ORE and not the HRE. FIG. 10A: Above, Equal amounts of MELAS cell lysates, treated with vehicle (DMSO) or increasing amounts of tizoxanide (2.5 to 40 μM) for 24 h, were separated on an SDS-PAGE gel and probed for HIF2α, MNRR1, and Tubulin. Below, Protein quantification for MNRR1 and HIF2α relative to Tubulin. FIG. 10B: Dual luciferase reporter assay showing relative activation of MNRR1-luciferase in MELAS cybrid cells overexpressing WT (FIG. 9A), Δ 801-952 (FIG. 9A), ΔORE (positions 47-53 of SEQ ID NO: 25 in FIG. 9C) or ΔHRE (positions 59-66 of SEQ ID NO: 25 in FIG. 9C). FIG. 10C: Dual luciferase reporter assay showing relative activation of HRE-luciferase levels in cybrid cells with 0% (CL9) and 70% MELAS heteroplasmy (DW7). FIG. 10D: Dual luciferase reporter assay showing relative activation of MNRR1− luciferase levels in MELAS cybrid cells with 0% heteroplasmy (CL9) overexpressing EV (empty vector) or HIF2α. FIG. 10E: Sequences in MNRR1 promoter highlighting the binding sites for RBPJk (positions 1-7 of SEQ ID NO: 26) and HIF2α (positions 5-9 of SEQ ID NO: 27) on opposite strands of DNA. In all figures, * indicates p<0.05, ** indicates p<0.005.

FIGS. 11A-11F: RBPJk and HIF2α compete for binding at the ORE in the MNRR1 promoter to regulate transcription. FIG. 11A: Chromatin immunoprecipitation-qPCR assessing binding of HIF2α to the endogenous MNRR1 promoter in MELAS cybrid cells with 0% (CL9) and 70% heteroplasmy (DW7). FIG. 11B: Dual luciferase reporter assay showing relative activation of MNRR1-luciferase levels in MELAS cybrid cells overexpressing varying proportions of constitutively active RBPJk (RBP-CA) and HIF2α. FIG. 11C: Dual luciferase reporter assay showing relative activation of MNRR1-luciferase levels in MELAS cybrid cells overexpressing the WT (FIG. 9A) or ΔORE (positions 47-53 in SEQ ID NO: 25 in FIG. 9C) promoter with EV (dotted line) or HIF2α. FIG. 11D: Equal amounts of Control or HIFα knockdown (KD) MELAS cybrid cell lysates were separated on an SDS-PAGE gel and probed for HIF2α and MNRR1 levels. Tubulin was probed as loading control. FIG. 11E: Oxygen consumption measured from Control or HIFα knockdown (KD) MELAS cybrid cells. (n=4 biological replicates, error bars represent SE). FIG. 11F: Left, Oxygen consumption measured from MELAS cybrid cells treated with vehicle (DMSO) or PT2385 (10 μM), an inhibitor of HIF2α function, for 24 h. Right, Equal amounts of MELAS cybrid cell lysates treated with vehicle (DMSO) or PT2385 (10 μM) for 24 h, separated on an SDS-PAGE gel, and probed for MNRR1 levels. GAPDH was probed as a loading control.

FIGS. 12A-12C: FIG. 12A: Dual luciferase reporter assay showing relative activation of MNRR1-luciferase levels in MELAS cybrid cells overexpressing varying proportions of empty vector (EV) and constitutively active RBPJk (RBP-CA). FIG. 12B: Dual luciferase reporter assay showing relative activation of MNRR1-luciferase levels in MELAS cybrid cells overexpressing an empty vector (EV) or constitutively active RBPJk (RBP-CA) and HIF2α. FIG. 12C: Dual luciferase reporter assay showing relative activation of COX412-luciferase levels in MELAS cybrid cells overexpressing an empty vector (EV), MNRR1, and MNRR1+Auranofin (0.5 μM).

FIG. 13: RT-PCR for measuring HIF2α levels. 18S rRNA was used as housekeeper for both analyses (n=3 biological replicates).

FIGS. 14A-14D: PHD3 levels are reduced in MELAS cybrid cells and enhanced by nitazoxanide to increase MNRR1 levels. FIG. 14A: Transcript levels of PHD1, 2, and 3 in MELAS cybrid cells harboring the levels of heteroplasmy shown (data from [6]). FIG. 14B: Protein levels of PHD3 in MELAS cybrid cells with 0% (CL9) and 70% heteroplasmy (DW7). GAPDH was probed as loading control. FIG. 14C: Equal amounts of MELAS cybrid cell lysates, treated with vehicle (DMSO), nitazoxanide, or tizoxanide (10 μM) for 24 h, were separated on an SDS-PAGE gel and probed for PHD1, PHD3, MNRR1, and Tubulin. FIG. 14D: Oxygen consumption measured from MELAS cybrid cells overexpressing PHD1 or PHD3. (n=4 biological replicates, error bars show SE).

### REFERENCE TO SEQUENCES/SEQUENCE LISTING

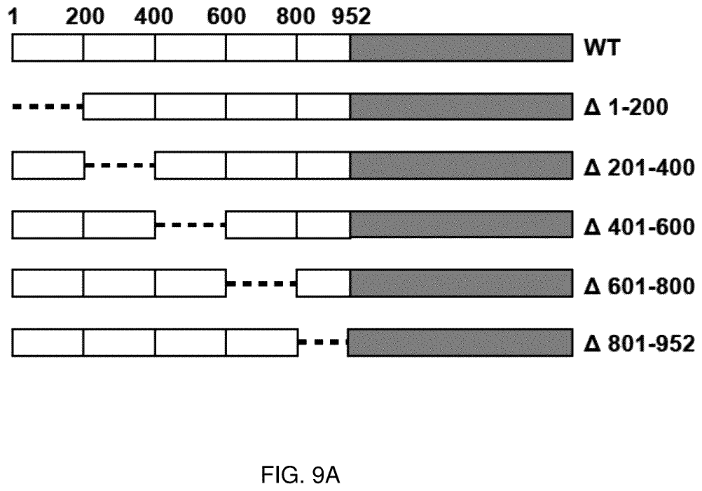

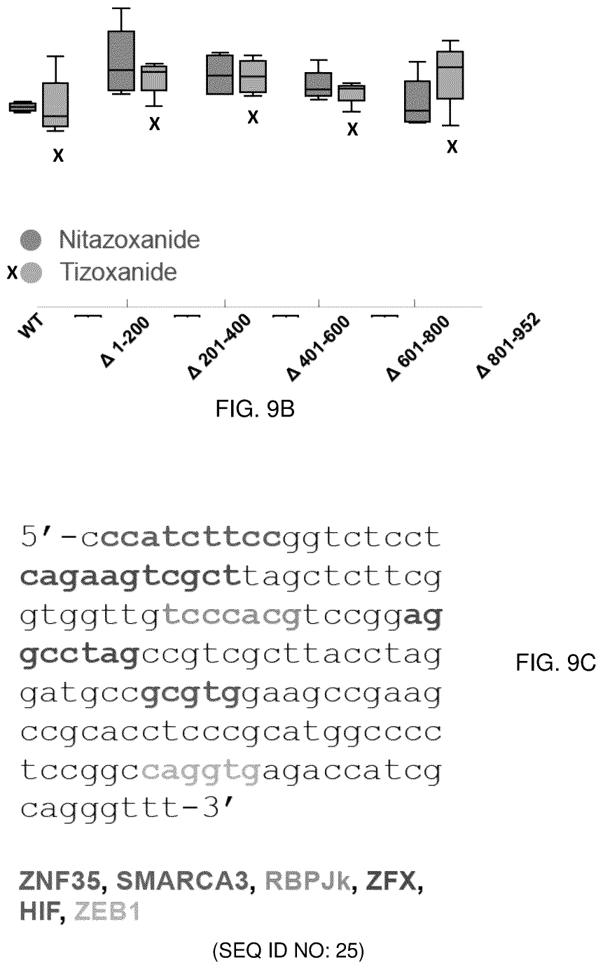

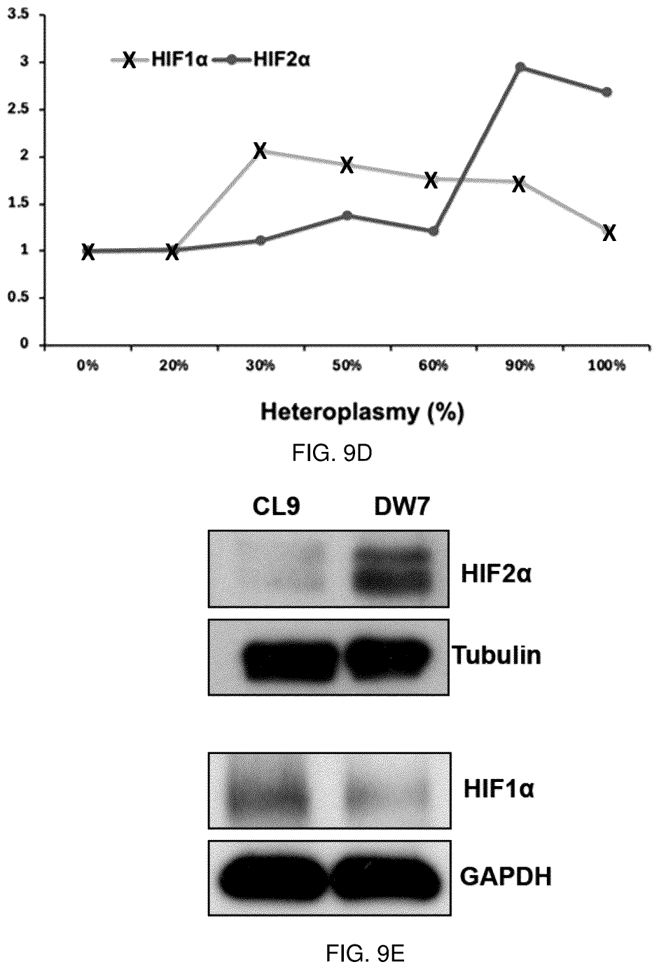

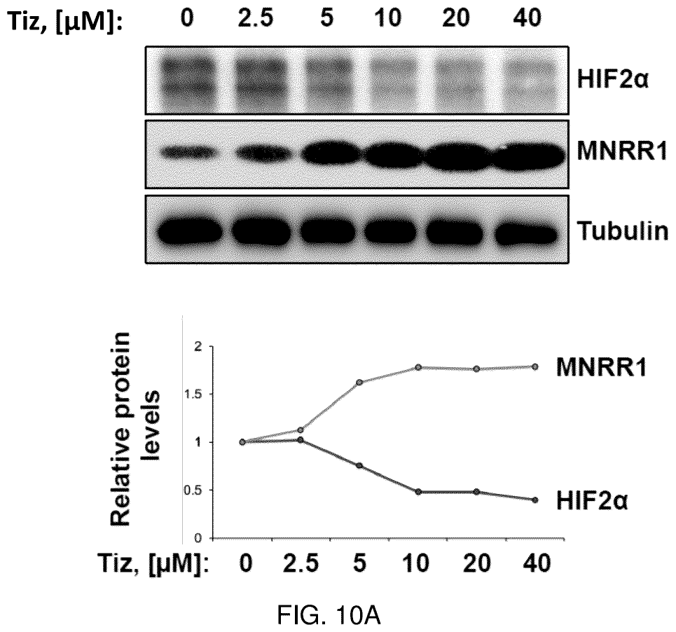

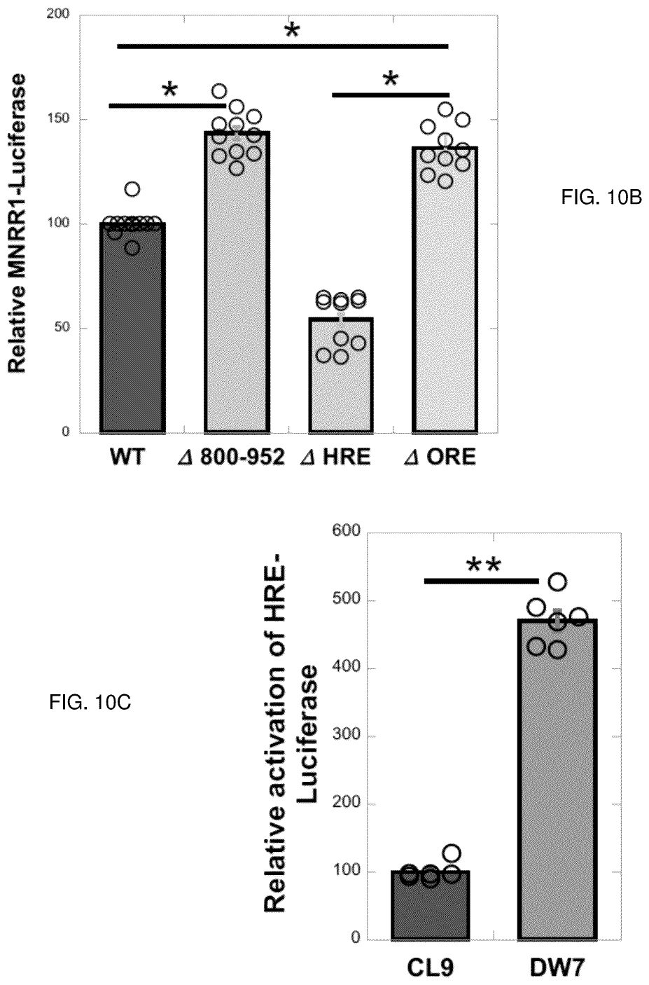

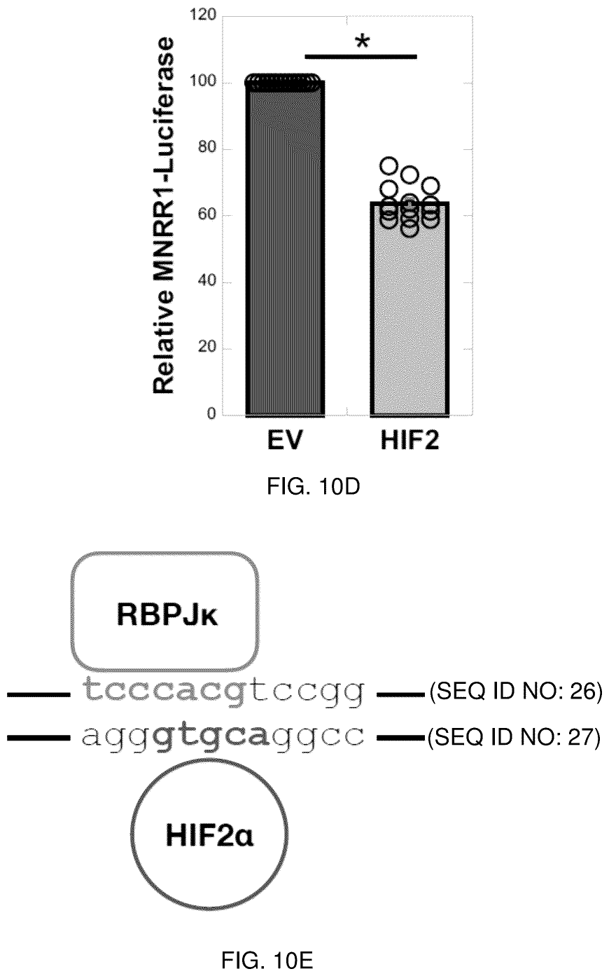

The nucleic acid and/or amino acid sequences described herein are shown using standard letter abbreviations, as defined in 37 C.F.R. § 1.822. Only one strand of each nucleic acid sequence is shown, but the complementary strand is understood as included in embodiments where it would be appropriate. In the Sequence Listing:
SEQ ID NO: 1 is a murine MNRR1 forward primer 5′-ATGGCCCAGATGGCTACC-3′
SEQ ID NO: 2 is a murine MNRR1 reverse primer 3′-CTGGTTCTGAGCACACTCCA-5′ (which is presented in the Sequence Listing in 5′ to 3′ order as: ACCTCACACGAGTCTTGGTC)
SEQ ID NO: 3 is a murine Actin forward primer 5′-TCCTCCCTGGAGAAGAGCTA-3′
SEQ ID NO: 4 is a murine Actin reverse primer 3′-ACGGATGTCAACGTCACACT-5′ (which is presented in the Sequence Listing in 5′ to 3′ order as: TCACACTGCAACTGTAGGCA)
SEQ ID NO: 5 is a human TNF forward primer 5′-TGTAGCAAACCCTCAAGCTG-3′
SEQ ID NO: 6 is a human TNF reverse primer 3′-GAGGTTGACCTTGGTCTGGT-5′ (which is presented in the Sequence Listing in 5′ to 3′ order as: TGGTCTGGTTCCAGTTGGAG)
SEQ ID NO: 7 is a human Actin forward primer 5′-CATTAAGGAGAAGCTGTGCT-3′
SEQ ID NO: 8 is a human Actin reverse primer 3′-GTTGAAGGTAGTTTCGTGGA-5′ (which is presented in the Sequence Listing in 5′ to 3′ order as: AGGTGCTTTGATGGAAGTTG)
SEQ ID NO: 9 is a MNRR1 forward primer 5′-CACACATGGGTCACGCCATTACT-3′
SEQ ID NO: 10 is a MNRR1 reverse primer 5′-TTCTGGGCACACTCCAGAAACTGT-3′
SEQ ID NO: 11 is a 18s forward primer 5′-CCAGTAAGTGCGGGTCATAA-3′
SEQ ID NO: 12 is a 18s reverse primer 5′-GGCCTCACTAAACCATCCAA-3′
SEQ ID NO: 13 is a PHD1(EGLN2) forward primer 5′-ACATCGAGCCACTCTTTGAC-3′
SEQ ID NO: 14 is a PHD1(EGLN2) reverse primer 3′-TCCTTGGCATCAAAATACC-5′ (which is presented in the Sequence Listing in 5′ to 3′ order as: CCATAAAACTACGGTTCCT)
SEQ ID NO: 15 is a PHD3(EGLN3) forward primer 5′-TCAAGGAGAGGTCTAAGGCAA-3′
SEQ ID NO: 16 is a PHD3(EGLN3) reverse primer 3′-ATGCAGGTGATGCAGCGA-5′ (which is presented in the Sequence Listing in 5′ to 3′ order as: AGCGACGTAGTGGACGTA)
SEQ ID NO: 17 is a HIF2α (EPAS1) forward primer 5′-CACCAAGGGTCAGGTAGTAA-3′
SEQ ID NO: 18 is a HIF2α (EPAS1) reverse primer 3′-AACACCACGTCATTCTTCTC-5′ (which is presented in the Sequence Listing in 5′ to 3′ order as: CTCTTCTTACTGCACCACAA)
SEQ ID NO: 19 is a mtDNA forward primer: 5′-CCTCCCTGTACGAAAGGAC-3′
SEQ ID NO: 20 is a mtDNA reverse primer: 5′-GCGATTAGAATGGGTACAATG-3′
SEQ ID NO: 21 is a GAPDH forward primer: 5′-GAGTCAACGGATTTGGTCGT-3′
SEQ ID NO: 22 is a GAPDH reverse primer: 5′-TTGATTTTGGAGGGATCTCG-3′
SEQ ID NO: 23 is a ChIP-qPCR forward primer: 5′-ATCTTCCGGTCTCCTCAGAA-3′
SEQ ID NO: 24 is a ChIP-qPCR reverse primer: 3′-AAACCCTGCGATGGTCTCA-5′ (which is presented in the Sequence Listing in 5′ to 3′ order as: ACTCTGGTAGCGTCCCAAA)
SEQ ID NO: 25 is the nucleic acid sequence of region of 800-952 bp in the MNRR1 promoter: (see FIG. 9C) CCCATCTTCCGGTCTCCTCAGAAGTCGCTTAGCTCTTCGGTG GTTGTCCCACGTCCGGAGGCCTAGCCGTCGCTTACCTAGGATGCCGCGTGGAAGCCGAA GCCGCACCTCCCGCATGGCCCCTCCGGCCAGGTGAGACCATCGCAGGGTTT. In the sequence, binding sites are found for the following transcription factors: ZNF35 (positions 2-10), SMARCA3 (positions 19-29), RBPJk (positions 47-53), ZFX (positions 59-66), HIF (positions 88-92), and ZEB1 (positions 130-135).
SEQ ID NO: 26 is the binding site for FBPJ_{K} within the MNRRQ promoter: 5′ TCCCACGTCCGG 3′ (see FIG. 10E); the binding site is a positions 1-7 of SEQ ID NO: 26.
SEQ ID NO: 27 is the binding site for HIF2α within the MNRRQ promoter: 3′ AGGGTGCAGGCC 5′ (see FIG. 10E; which is shown in the Sequence Listing in 5′ to 3′ order as: CCGGACGTGGGA); the binding site is a positions 5-9 of SEQ ID NO: 27.

### DETAILED DESCRIPTION

Aspects of the current disclosure are now described with additional details and options as follows: (1) Conditions to treat with MNRR1 Activators (II) MNRR1 Activators; (Ill) Compositions; (IV) Methods of Use; (V) Exemplary Embodiments; (VI) Experimental Examples; and (VII) Closing Paragraphs. These headings do not limit the interpretation of the disclosure and are provided for organizational purposes only.

### (I) Conditions to Treat with MNRR1 Activators

Conditions that may be treated through MNRR1 activations include inflammatory disorders, and more generally diseases or conditions that involve or are mediated (at least in part) by inflammation. One of ordinary skill in the art will recognize there are myriad inflammatory disorders, including conditions or disorders involving acute or chronic.

For instance, as described in U.S. Pat. No. 11,065,218, inflammation involves the activation of the immune system in response to harmful stimuli, such as, e.g., a pathogen, infection, irritant, or damage to cells. As a stereotyped response, inflammation is a mechanism of innate immunity, as compared to adaptive immunity, which is specific for each pathogen. Inflammation can be classified as either acute or chronic. Generally speaking, acute inflammation is mediated by granulocytes, while chronic inflammation is mediated by mononuclear cells such as monocytes and lymphocytes.

Acute inflammation is an initial protective response of the body to remove an injurious stimulus by maintaining tissue integrity and contributing to tissue repair. It a part of the body's natural defense system against injury and disease, and in the absence of acute inflammation, wounds and infections would never heal and progressive destruction of the tissue would compromise the survival of the organism.

The process of acute inflammation is initiated by cells already present in all tissues, mainly resident macrophages, dendritic cells, histiocytes, Kupffer cells, mastocytes, vascular endothelial cells, and vascular smooth muscle cells. At the onset of a harmful stimulus, these cells undergo activation and release inflammatory mediating and sensitizing molecules, such as, e.g., pro-inflammatory cytokines, pro-inflammatory prostaglandins, leukotrienes, histamine, serotonin, neutral proteases, bradykinin, and nitric oxide. These inflammatory molecules modulate a complex series of biological events involving cellular and acellular components of the local vascular system, the immune system, and the injured tissue site to propagate and mature the inflammatory response. These events are responsible for eliciting an acute inflammatory response, typically characterized by 1) vasodilatation which increases blood flow into the tissue thereby causing erythema (redness and warmth), which may extend beyond this site (the flare response); 2) blood vessel permeability which increases plasma leakage into the tissue thereby causing edema (swelling); 3) alter the excitability of certain sensory neurons causing hypersensitivity and pain; 4) stimulate the release of inflammation inducing molecules such as, e.g., neuropeptides like substance P (SP) and calcitonin gene-related peptide (CGRP), prostaglandins, and amino acids like glutamate, from the peripheral nerve endings; and 5) increase migration of leukocytes, mainly granulocytes, from the blood vessels into the tissue. An acute inflammatory response requires constant stimulation to be sustained and must be actively terminated when no longer needed. Hence, acute inflammation ceases once the injurious stimulus has been removed.

However, severe or prolonged noxious stimulation results in a chronic inflammatory response that leads to a progressive shift in the type of cells present at the site of tissue injury. Chronic inflammation may be characterized as the simultaneous destruction and healing of tissue from the inflammatory process, with the net result of provoking injury rather than mediating repair. As such, chronic inflammation is a disease. As an inflammatory response can occur anywhere in the body, chronic inflammation has been implicated in the pathophysiology of a wide range of seemingly unrelated disorders which underlay a large and varied group of human diseases. For example, chronic inflammation is involved in diseases as diverse as cardiovascular diseases, cancers, allergies, obesity, diabetes, digestive system diseases, degenerative diseases, auto-immune disorders, and Alzheimer's disease.

It is known that inflammation can be influenced by mitochondrial constituents and metabolic products (see, e.g., Marchi et al., Nat Rev Immunol. 23(3):159-173, 2023).

Specific exemplary inflammatory disorders include inflammation-induced preterm labor and inflammation-induced preterm birth. Preterm births or premature births are births that occur before gestation time that considered full term birth. In humans, preterm births are births that occur before 37 weeks of gestation.

Inflammation-induced preterm labor and inflammation-induced preterm birth may be caused by microbial infections.

### (II) MNRR1 Activators

Nitazoxanide is shown herein to be effective to increase MNRR1 activity (by stimulating MNRR1 transcription), and with that to prevent/reverse inflammation-induced preterm delivery (exemplified using an LPS-based mouse model of intra-amniotic inflammation).

Nitazoxanide is commercially available under a number of name brands (including Adonid, Alinia, Allpar, Annita, Celectan, Colufase, Daxon, Dexidex, Diatazox, Kidonax, Mitafar, Nanazoxid, Parazoxanide, Netazox, Niazid, Nitamax, Nitax, Nitaxide, Nitaz, Nizonide, NT-TOX, Pacovanton, Paramix, Toza, and Zox); it is also available as a generic drug. It is a broad-spectrum antiparasitic and broad-spectrum antiviral medication that is used in medicine for the treatment of various helminthic, protozoal, and viral infections. It is appropriate for use in treating infection by Cryptosporidium parvum and Giardia lamblia, including in immunocompetent individuals. It has also been repurposed for the treatment of influenza. Some studies show that nitazoxanide has in vitro antiparasitic activity; it may have efficacy in treating infections caused by other protozoa and helminths as well as a number of viral infections (including hepatitis).

Chemically, nitazoxanide is a prototypical thiazolide, a class of synthetic nitrothiazolyl-salicylamide derivatives with antiparasitic and antiviral activity. Tizoxanide, an active metabolite of nitazoxanide in humans, is also an antiparasitic drug of the thiazolide class.

Nitazoxanide (NTZ) has the following chemical formula:

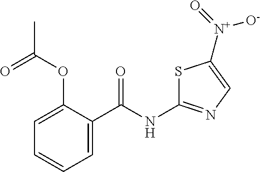 [SMILES: CC(=O)Oc1ccccc1C(=O)Nc1ncc([N+](=O)[O-])s1]

As described in the Drugbank Online entry for nitazoxanide (DB00507), The general effect of this medication is the prevention of microbe activity through disruption of important energy pathways for survival and proliferation. Nitazoxanide exhibits antiprotozoal activity by interfering with the pyruvate ferredoxin/flavodoxin oxidoreductase (PFOR) dependent electron transfer reaction, an essential reaction needed for anaerobic energy metabolism of various microorganisms. Sporozoites of Cryptosporidium parvum and trophozoites of Giardia lamblia are therefore inhibited, relieving symptoms of diarrhea. Interference with the PFOR enzyme-dependent electron transfer reaction may only be one of the many pathways by which nitazoxanide exhibits antiprotozoal activity. In parasitic-protozoa, NTZ also induces lesions in the cell membranes and depolarizes the mitochondrial membrane while inhibiting quinone oxidoreductase NQO1, nitroreductase-1 and protein disulphide isomerase enzymes. In addition, this drug inhibits the glutathione-S-transferase (a major detoxifying enzyme) and modulates the Avr-14 gene, which encodes the alpha-type subunit of glutamate-gated chloride ion channel present in nematodes.

Aside from its well understood non-competitive inhibition of the PFOR in anaerobic bacteria, NTZ also demonstrates various other antibacterial mechanisms. It inhibits pyruvate dehydrogenase in E. coli, disrupts the membrane potential and pH homeostasis in the Mycobacterium tuberculosis, suppresses the chaperone/usher (CU) pathway of the gram-negative bacteria, and stimulates host macrophage autophagy in tuberculosis patients. NTZ also suppresses viral replication by inhibiting the maturation of the viral hemagglutinin and the viral transcription factor immediate early 2 (IE2) as well as by activating the eukaryotic translation initiation factor 2a (an antiviral intracellular protein). NTZ exhibits an inhibitory effect on tumor cell progression by altering drug detoxification (glutathione-S-transferase P1), unfolded protein response, autophagy, anti-cytokines activity, and c-Myc inhibition.

For additional information, see: Anderson & Curran (Drugs 67(13)1947-1967, 2007), Shakya et al. (Curr Drug Discov Technol. 2017, 10.2174/1570163814666170727130003), Rossignol (Antiviral Res 11094-103, 2014), Balamurugan & Said (Am J Physiol Cell Physiol, 291(1): C189-193, 2006), Broekhuyser et al., Int J Clin Pharmacol Ther 38(8):397-394, 2000; Pub Chem “Nitrazoxanide”, EPA Chemistry Dashboard “Nitazoxanide”, FDA Approval of Alinia@nitazoxanide (NDA Number 21-497, Jan. 28, 2004).

Notably, no target NTZ has been identified in humans.

More generally, MNRR1 activators include compounds that comprise at least one active tizoxanide moiety. Additional MNRR1 activator compounds, and methods for identifying additional MNRR1 activators, are also described.

### (III) Compositions

The MNRR1 activators/MNRR1 activating agents (e.g., exemplified by nitazoxanide) useful in methods disclosed herein can be formulated into compositions for direct administration to a subject.

Commercially available preparations of nitazoxanide, for instance formulated for use in anti-parasitic and antiviral treatment methods, can be used in the methods describe herein. Notably, commercially available preparations of nitazoxanide include orally acceptable formulations, which have the benefit of simple administration and good tolerability in treated subjects.

The compositions disclosed herein can be formulated for administration by injection, inhalation, infusion, perfusion, lavage, or ingestion. The compositions disclosed herein can further be formulated for infusion via catheter, intravenous, intramuscular, intratumoral, intradermal, intraarterial, intranodal, intralymphatic, intraperitoneal, intralesional, intraprostatic, intravaginal, intrarectal, topical, intrathecal, intraparenchymal, intravesicular, intracerebroventricular, subpial, intravitreal, subretinal, transgastric, transduodenal, intracystic, bronchoscopic, aerosol, oral and/or subcutaneous administration.

For injection and infusion, compositions can be formulated as aqueous solutions, such as in buffers including Hanks' solution, Ringer's solution, or physiological saline. The aqueous solutions can contain formulatory agents such as suspending, stabilizing and/or dispersing agents. Alternatively, the formulation can be in lyophilized and/or powder form for constitution with a suitable vehicle, e.g., sterile pyrogen-free water, before use.

Any composition or formulation disclosed herein can advantageously include any other pharmaceutically acceptable carriers which include those that do not produce significantly adverse, allergic, or other untoward reactions that outweigh the benefit of administration, whether for research, prophylactic and/or therapeutic treatments. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety and purity standards as required by United States FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants. In particular embodiments, a pharmaceutically acceptable carrier includes buffered saline. Buffered saline can include exemplary buffering agents described herein.

Exemplary buffering agents include citrate buffers, succinate buffers, malate buffers, tartrate buffers, fumarate buffers, gluconate buffers, aspartate buffers, glutamate buffers, α-ketoglutarate buffers, oxalate buffers, lactate buffers, acetate buffers, phosphate buffers, histidine buffers and/or trimethylamine salts.

Exemplary preservatives include phenol, benzyl alcohol, meta-cresol, methyl paraben, propyl paraben, octadecyl dimethyl benzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol, or mannitol. Exemplary stabilizers include organic sugars, polyhydric sugar alcohols, polyethylene glycol; sulfur-containing reducing agents, amino acids, low molecular weight polypeptides, proteins, immunoglobulins, hydrophilic polymers, or polysaccharides.

Compositions can also be formulated as depot preparations, including for instance where long-term administration is desired (such as for treatment of chronic inflammation). Depot preparations can be formulated with suitable polymeric or hydrophobic materials (for example as an emulsion in an acceptable oil) or ion exchange resins, or as sparingly soluble derivatives, for example, as sparingly soluble salts.

Additionally, compositions can be formulated as sustained-release systems, including for instance where long-term administration is desired (such as for treatment of chronic inflammation), utilizing semipermeable matrices of solid polymers containing at least one active ingredient. Various sustained-release materials have been established and are well known by those of ordinary skill in the art. Sustained-release systems may, depending on their chemical nature, release active ingredients following administration for two weeks to 1 month. In particular embodiments, a sustained-release system could be utilized, for example, if a human patient were to miss a weekly administration.

### (IV) Methods of Use

The formulations disclosed herein can be used for treating subjects, including humans, veterinary animals (dogs, cats, reptiles, birds, etc.), livestock (horses, cattle, goats, pigs, chickens, etc.), and research animals (monkeys, rats, mice, fish, etc.). In particular embodiments, a subject is a human patient having or suspected of having a disease characterized at least in part by inflammation, and/or low MNRR1 activity (compared to a healthy individual). Treating subjects includes delivering therapeutically effective amounts. Therapeutically effective amounts include those that provide effective amounts, prophylactic treatments, and/or therapeutic treatments.

An “effective amount” is the amount of a pharmaceutical composition including nitazoxanide that is sufficient to generate a desired response, such as increasing an activity of MNRR1 and/or decreases inflammation. Effective amounts may be administered for research purposes.

A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition. A “therapeutic treatment” includes a treatment administered to a subject who displays symptoms or signs of a condition (such as a condition mediated at least in part by inflammation) and is administered to the subject for the purpose of reducing the severity or progression of the condition.

The actual dose and amount of composition(s) including nitazoxanide, administered to a particular subject can be determined by a physician, veterinarian, or researcher taking into account parameters such as physical and physiological factors including target; body weight; type of condition; severity of condition; upcoming relevant events, when known; previous or concurrent therapeutic interventions; idiopathy of the subject; and route of administration, for example. In addition, in vitro and in vivo assays can optionally be employed to help identify optimal dosage ranges.

Therapeutically effective amounts of compositions described herein to administer can include doses ranging from, for example, 0.5 mg/kg to 25 mg/kg, or 1 mg/kg to 20 mg/kg. In particular embodiments, doses can include 0.5 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 17 mg/kg, 17.5 mg/kg, 18 mg/kg, 18.5 mg/kg, 19 mg/kg, 19.5 mg/kg, 20 mg/kg, or greater. In particular embodiments, doses can include 50 mg/m² to 1000 mg/m², or 100 mg/m² to 800 mg/m². In particular embodiments, doses can include 50 mg/m², 100 mg/m², 150 mg/m², 200 mg/m², 250 mg/m², 300 mg/m², 350 mg/m², 400 mg/m², 450 mg/m², 500 mg/m², 550 mg/m², 600 mg/m², 650 mg/m², 700 mg/m², 750 mg/m², 800 mg/m², or greater.

The current use of nitazoxanide in the clinics for parasitic infections is 500 mg (per adult) twice a day. This translates to around 600 micromolar drug concentration in the blood. This standard dosage of 500 mg twice daily is useful with the currently described methods as well.

A single dose of 4 gm of the drug did not result in any adverse effects in adult humans.

Therapeutically effective amounts can be administered through any appropriate administration route such as by injection, infusion, lavage, and beneficially by oral administration.

Also contemplated are combination therapy, in which the herein described treatment with nitazoxanide (or another agent that activates MNRR1) is combined (concurrently or sequentially) with another treatment to reduce the likelihood of preterm labor or preterm (premature) birth. By way of example, such additional treatment(s) include bed rest, cerclage, and hormone treatment (that is, treatment of the pregnant subject with progesterone). See, for instance, Meis et al. (NEJM, 348, 2379-2385, 2003); ACOG Practice Bulletin, Number 234 (Obstet Gynecol 138(2): e65-e90, 2021; doi: 10.1097/AOG.0000000000004479); Shennan et al. (Int. J. Gynaecol Obstet 155(1):16-18, 2021); Lucovnik et al. (Acta Obstet Gynecol Scand. 90(10):1057-2069, 2011); and Romero et al. (Semin Fetal Neonatal Med. 15(9):15-26, 2013).

The Exemplary Embodiments and Example(s) below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (V) Exemplary Embodiments

### First Set of Exemplary Embodiments

1. A method of delaying inflammation-induced labor in a pregnant subject, including: selecting a pregnant subject; and activating MNRR1 in a cell of the pregnant subject.

2. The method of embodiment 1, wherein the cell is a placental cell.

3. The method of embodiment 1, wherein activating MNRR1 in the cell of the pregnant subject includes administering an effective amount of nitazoxanide to the pregnant subject.

4. The method of embodiment 3, wherein the nitazoxanide is administered prior to exposure of the pregnant subject to an inflammation-causing agent.

5. The method of embodiment 4, wherein the inflammation-causing agent includes a microbe.

6. The method of embodiment 3, wherein the administering is oral administration.

7. The method of embodiment 1, wherein delaying inflammation-induced labor in the pregnant subject prevents premature birth.

### Second Set of Exemplary Embodiments

1. A method of delaying inflammation-induced labor in a pregnant subject, including: selecting a pregnant subject; and activating MNRR1 in a cell of the pregnant subject.

2. The method of embodiment 1, wherein the cell is a placental cell.

3. The method of embodiment 1, wherein activating MNRR1 in the cell of the pregnant subject includes administering an effective amount of a MNRR1 activating agent to the pregnant subject.

4. The method of embodiment 3, wherein the MNRR1 activating agent includes at least one active tizoxanide moiety.

5. The method of embodiment 3, wherein the MNRR1 activating agent includes nitazoxanide.

6. The method of embodiment 3, wherein the MNRR1 activating agent is administered prior to exposure of the pregnant subject to an inflammation-causing agent.

7. The method of embodiment 6, wherein the inflammation-causing agent includes a microbe.

8. The method of embodiment 3, wherein the administering is oral administration.

9. The method of embodiment 1, wherein delaying inflammation-induced labor in the pregnant subject prevents premature birth.

10. The method of embodiment 1, optionally including administering an effective amount of progesterone to the pregnant subject.

11. The method of embodiment 5, wherein the nitazoxanide is administered to the pregnant subject in a dose of 500 mg twice daily.

12. A method of reducing or preventing pre-term labor in a pregnant subject, including: selecting a pregnant subject; and administering a MNRR1 activating agent to the pregnant subject.

13. The method of embodiment 12, wherein the MNRR1 activating agent includes at least one active tizoxanide moiety.

14. The method of embodiment 12, wherein the MNRR1 activating agent includes nitazoxanide, optionally wherein the nitazoxanide is administered to the pregnant subject in a dose of 500 mg twice daily.

15. The method of embodiment 12, wherein the MNRR1 activating agent is administered prior to exposure of the pregnant subject to an inflammation-causing agent.

16. The method of embodiment 15, wherein the inflammation-causing agent includes a microbe.

17. The method of embodiment 12, wherein the administering is oral administration.

18. The method of embodiment 12, wherein reducing or preventing pre-term labor in the pregnant subject prevents premature birth.

19. The method of embodiment 12, optionally including administering an effective amount of progesterone to the pregnant subject.

20. A method of treating an inflammatory disorder or a mitochondria-associated disorder in a subject, including activating Mitochondrial Nuclear Retrograde, Regulator 1 (MNRR1) in a cell of the subject.

### (VI) Experimental Examples

### Example 1: Anti-Inflammatory Effects of Nitazoxanide are Mediated Via Transcriptional Activation of MNRR1

Given the observed beneficial effects of restoring MNRR1 expression in vitro, a screen of FDA-approved drugs to identify pharmacological activators of MNRR1 was performed and identified was nitazoxanide as an inducer of MNRR1 transcription. Moreover, it was shown that nitazoxanide could rescue the trophoblast phenotype induced by LPS treatment (Purandare et al., iScience, 25: 105342, 2022). Therefore, in the current study (published as Purandare et al., Placenta 140:66-71, 8 Jul. 2023 and Purandare et al., bioRxiv, 1 Feb. 2023), whether nitazoxanide could represent a viable strategy for preventing preterm birth induced by intra-amniotic inflammation caused by the microbial product LPS was examined.

### Materials and Methods

Cell lines: All cell media were supplemented with 5% fetal bovine serum (FBS) (Sigma Aldrich, St. Louis, MO, USA) plus Penicillin-Streptomycin (HyClone, Logan, UT, USA). HTR8/SVneo (HTR) cells were cultured in Roswell Park Memorial Institute Medium (RPMI) (HyClone, Logan, UT, USA).

Chemicals: Nitazoxanide was obtained from Selleckchem (Houston, TX, USA) and solubilized in DMSO (used as vehicle control in all cell culture nitazoxanide experiments). Alinia® was obtained from All Care Pharmacy (Allen Park, MI, USA). Ultrapure LPS for cell culture experiments (lipopolysaccharide from Escherichia coli 0111: B4) was purchased from Invivogen (San Diego, CA, USA).

Plasmids: The MNRR1 promoter firefly luciferase reporter plasmid and pRL-SV40 (Renilla) luciferase plasmids (for normalization of firefly luciferase) have been described previously (Aras et al., Mitochondrion, 20:43-51, 2015). All plasmids were purified by using the EndoFree plasmid purification kit from Qiagen (Germantown, MD, USA).

Transfections and luciferase reporter assays: HTR cells were transfected with the indicated plasmids, using TransFast transfection reagent (Promega, Madison, WI, USA) according to the manufacturer's protocol as previously described (Purandare et al., iScience, 25: 105342, 2022). Luciferase assays were performed with the dual-luciferase reporter assay kit (Promega, Madison, WI, USA). Transfection efficiency for the MNRR1 reporter was normalized with the co-transfected pRL-SV40 Renilla luciferase expression plasmid (Aras et al., Nucleic Acids Res., 41:2255-2266, 2013; Purandare et al., J. Biol. Chem., 293:6517-6529, 2018; Aras et al., Biochim. Biophys. Acta Mol. Cell Res., 1864:440-448, 2017). EC₅₀ was calculated by using the Quest Graph™ EC₅₀ Calculator (AAT Bioquest, Inc., on the World Wide Web at aatbio.com/tools/ec50-calculator).

Real-time polymerase chain reaction: Total cellular RNA was extracted from mouse placental tissues or HTR8 cells by using a RNeasy Plus Mini Kit (Qiagen, Germantown, MD, USA), according to the manufacturer's instructions. Complementary DNA (cDNA) was generated by reverse transcriptase polymerase chain reaction (PCR) with the ProtoScript® II First Strand cDNA Synthesis Kit (New England Biolabs, Ipswich, MA, USA). Transcript levels were measured by real-time PCR by using SYBR green on an ABI 7500 system. Real-time analysis was performed by the ΔΔ^{Ct} method as previously described (Purandare et al., J. Biol. Chem., 293:6517-6529, 2018). The primer sequences were as follows:

MNRR1 mouse (forward)
(SEQ ID NO: 1)
5′-ATGGCCCAGATGGCTACC-3′,
MNRR1 mouse (reverse)
(SEQ ID NO: 2)
3′-CTGGTTCTGAGCACACTCCA-5′;
Actin mouse (forward)
(SEQ ID NO: 3)
5′-TCCTCCCTGGAGAAGAGCTA3′,
Actin mouse (reverse)
(SEQ ID NO: 4)
3′-ACGGATGTCAACGTCACACT-5′;
TNF human (forward)
(SEQ ID NO: 5)
5′-TGTAGCAAACCCTCAAGCTG-3′,
TNF human (reverse)
(SEQ ID NO: 6)
3′-GAGGTTGACCTTGGTCTGGT-5′;
Actin human (forward)
(SEQ ID NO: 7)
5′-CATTAAGGAGAAGCTGTGCT-3′,
and
Actin human (reverse)
(SEQ ID NO: 8)
3′-GTTGAAGGTAGTTTCGTGGA-5′.

Immunoblotting: Immunoblotting was performed as previously described (Aras et al., Nucleic Acids Res., 41:2255-2266, 2013; Aras et al., Mitochondrion, 20:43-51, 2015). Tissue lysates were prepared by using 50 mM Tris-HCl (pH 8.0) with 150 mM NaCl, 1% NP-40, 0.5% sodium deoxycholate, and 0.1% SDS. All lysis buffers included a protease and phosphatase inhibitor cocktail (Sigma, PPC1010).

Cytokine array: Cell culture supernatants were centrifuged at 1200 rpm to pellet cells and supernatants were used with a membrane array to measure cytokines and chemokines. To do so, the supernatant was collected, mixed with fresh 100% trichloroacetic acid solution in a 4:1 ratio, and incubated for 1 h at 4° C. After centrifugation at 14,000 rpm for 30 min at 4° C., the pellet was collected, washed twice with acetone, and centrifuged as above but for only 10 min. The pellet was air-dried, resuspended in array buffer, and again collected by centrifugation. The supernatant was then used for the array per manufacturer's instructions (Proteome Profiler Array Catalog #ARY005B, Biotechne, Minneapolis, MN, USA). The sample in the bottom right corner is blank (a negative control), and the remaining three corner samples are proprietary positive controls.

Mice: C57BL/6J mice were purchased from The Jackson Laboratory (Bar Harbor, ME, USA), and bred in the animal care facility at the C.S. Mott Center for Human Growth and Development at Wayne State University (Detroit, MI, USA). Mice were housed under a circadian cycle (12 h light:12 h dark). Eight- to 12-week-old females were mated with males of proven fertility. Female mice were examined daily between 8:00 a.m. and 9:00 a.m. for the presence of a vaginal plug, which indicated 0.5 days post coitum (dpc). Female mice with a vaginal plug were removed from the mating cages and housed separately. A weight gain of >2 g confirmed pregnancy at 12.5 dpc. All animal experiments were approved by the Institutional Animal Care and Use Committee at Wayne State University (Protocol No. 21-04-3506).

Animal model of intra-amniotic LPS-induced preterm labor and birth: Nitazoxanide (Alinia®; ROMARK, Tampa, FL, USA) was dissolved in sterile injectable water. The nitazoxanide volume was mouse weight dependent (100 μL of nitazoxanide solution at 200 mg/kg was administered for every mouse weight of 10 g). Control mice did not receive carrier solution based on previous validation studies showing no effect of gavage intervention on preterm birth or neonatal mortality. Dams were anesthetized on 16.5 dpc by inhalation of 2-3% (Fluriso™ (Isoflurane, USP), Vetone Boise, ID, USA) and 1-2 L/min of oxygen in an induction chamber. Anesthesia was maintained with a mixture of 1.5-2% isoflurane and 1.5-2 L/min of oxygen. Dams were positioned on a heating pad and stabilized with adhesive tape. Fur removal from the abdomen and thorax was performed by applying Nair cream (Church & Dwight Co., Inc., Ewing, NJ, USA) to those areas. Body temperature was detected with a rectal probe (VisualSonics Inc., Toronto, Ontario, Canada) and maintained in the range of 37±1° C. Respiratory and heart rates were monitored by electrodes embedded in the heating pad. An ultrasound probe was fixed and mobilized with a mechanical holder, and the transducer was slowly moved toward the abdomen. Ultrasound-guided intra-amniotic injection of lipopolysaccharide of Escherichia coli 0111: B4 (LPS; Sigma-Aldrich, St. Louis, MO, USA) at a concentration of 100 ng dissolved in 25 μL of sterile 1× PBS was performed into each amniotic sac by using a 30 G needle (BD PrecisionGlide Needle), and controls were injected with 25 μL of sterile 1× PBS alone. The syringe was stabilized by a mechanical holder (VisualSonics Inc.). Following ultrasound, dams were placed under a heat lamp for recovery (dam regaining normal activity, such as walking and responding), which occurs 10-15 min after removal from anesthesia. Following injection, dams were monitored by using a video camera with infrared light (Sony) until delivery. Video monitoring allowed for determination of gestational length, which was calculated from the presence of the vaginal plug (0.5 dpc) until the observation of the first pup in the cage bedding. Preterm birth was defined as delivery before 18.5 dpc, and its rate was represented by the percentage of dams delivering preterm among the total number of mice injected. The rates of neonatal mortality at birth were calculated as the number of pups found dead among the total litter size.

Nitazoxanide treatment: Dams were treated with 200 mg/kg of nitazoxanide (Alinia®, ROMARK, Tampa, FL, USA) via oral gavage at 8 p.m. on 15.5 dpc, again at 8 a.m., 12 p.m., 4 p.m., and 8 p.m. on 16.5 dpc, and then the tissues were collected the morning of 17.5 dpc for molecular experiments. These dams were intra-amniotically injected with either LPS or PBS control at 10 a.m. on 16.5 dpc.

High-throughput RT qPCR: This was performed as previously described (Motomura et al., JCI Insight, 7, 2022). Briefly, total RNA was isolated from decidua with RNeasy Mini Kits (Qiagen), according to the manufacturer's instructions. RNA concentrations, purity, and integrity were evaluated with the NanoDrop 8000 spectrophotometer (Thermo Scientific, Wilmington, DE, USA) and the Bioanalyzer 2100 (Agilent Technologies, Wilmington, DE, USA). cDNA was synthesized by using SuperScript IV VILO Master Mix (Invitrogen by Thermo Fisher Scientific Baltics UAB, Vilnius, Lithuania). Gene expression profiling of the tissues was performed on the BioMark System for high-throughput RT-qPCR (Fluidigm, San Francisco, CA, USA) or the ABI 7500 Fast real-time PCR system (Applied Biosystems, Life Technologies Corporation, Pleasanton, CA, USA) with the TaqMan gene expression assays (Applied Biosystems, Life Technologies Corporation). This list has been provided as Table 1. Data were normalized by using multiple reference genes (Gusb, Hsp90abl, Gapdh, and Actb) averaged within each sample, and relative changes in transcript levels were calculated by the AACt method. Heatmaps were plotted in Excel (Microsoft, Redmond, WA, USA).

TABLE 1

| Name | Symbol | Assay ID |
|---|---|---|
| Actin, beta | Adtb | Mm04394036_g1 |
| Glucuronidase, beta | Busb | Mm01197698_m1 |
| Glyceraldehyde-3-phosphate | Gadph | Mm99999915_g1 |
| dehydrogenase | | |
| HSP 90 alpha (cytosolic), | Hsp90ab1 | Mm00833431_g1 |
| class B member 1 | | |
| Interleukin-1 beta | II1b | Mm00434228_m1 |
| Interleukin-2 | II6 | Mm00446190_m1 |
| Interleukin-6 | II10 | Mm01288386_m1 |
| Interferon gamma | Ifng | Mm01168134_m1 |
| Toll-like receptor 4 | Tlr4 | Mm00445273_m1 |
| Toll-like receptor 9 | Tlr9 | Mm00446193_m1 |
| NLR Family, pyrin domain | Nlrp3 | Mm00840904_m1 |
| containing 3 | | |
| Tumor necrosis factor | Tnf | Mm00443258_m1 |
| Prostaglandin-endoperoxide | Ptgs2 | Mm00478374_m1 |
| synthase 2 | | |

Statistical analyses: Statistical analyses were performed with MSTAT version 6.1.1 (N. Drinkwater, University of Wisconsin, Madison, WI). The two-sided Wilcoxon rank-sum test was applied to determine statistical significance for p-values. Data were considered statistically significant with * for p<0.05 and ** for p<0.01. Error bars represent standard error of mean.

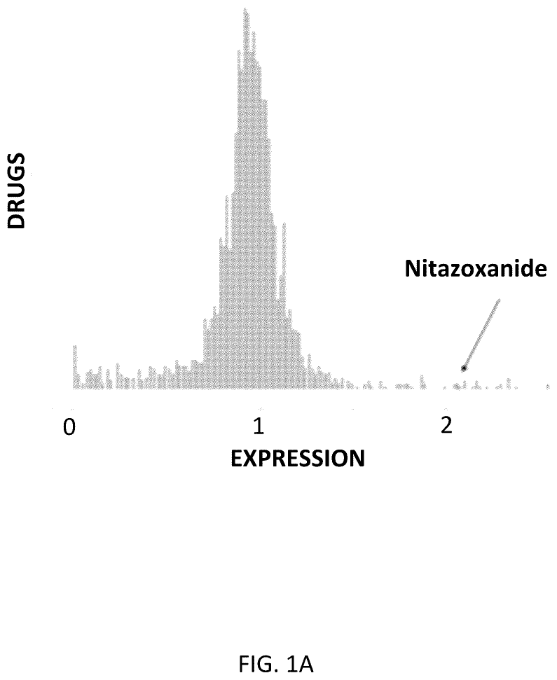

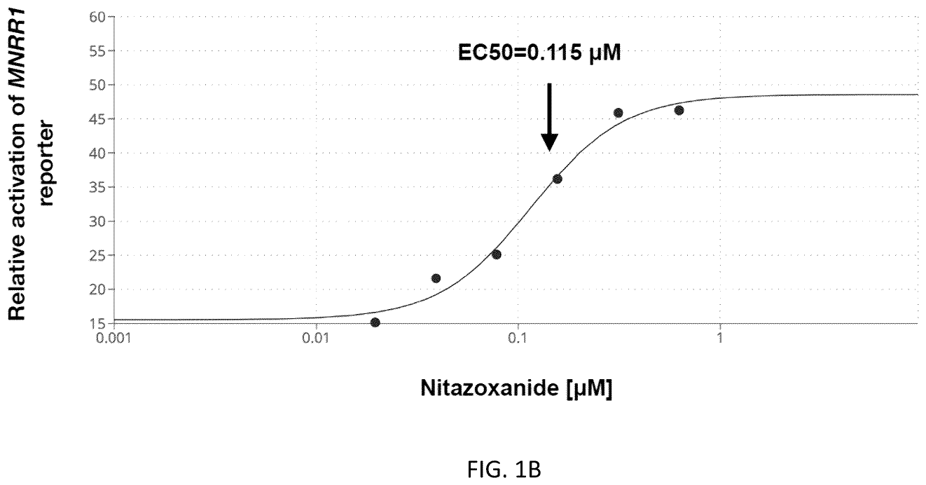

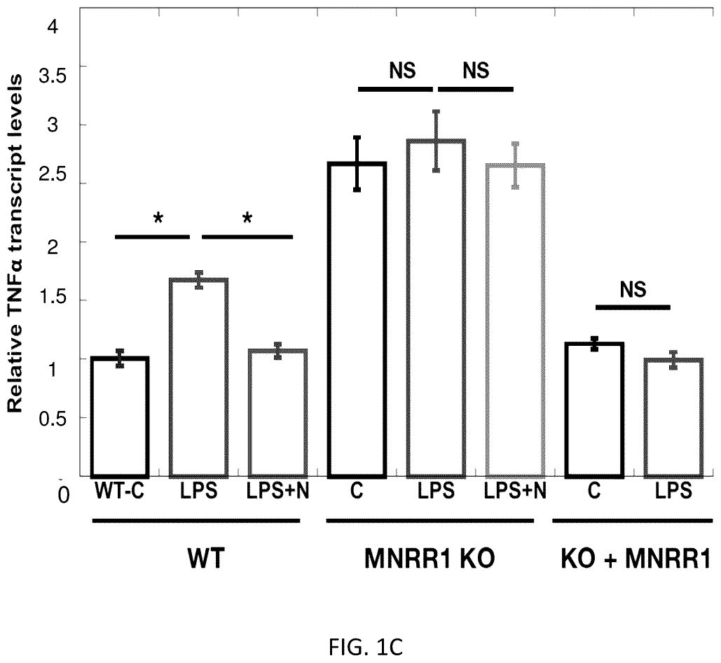

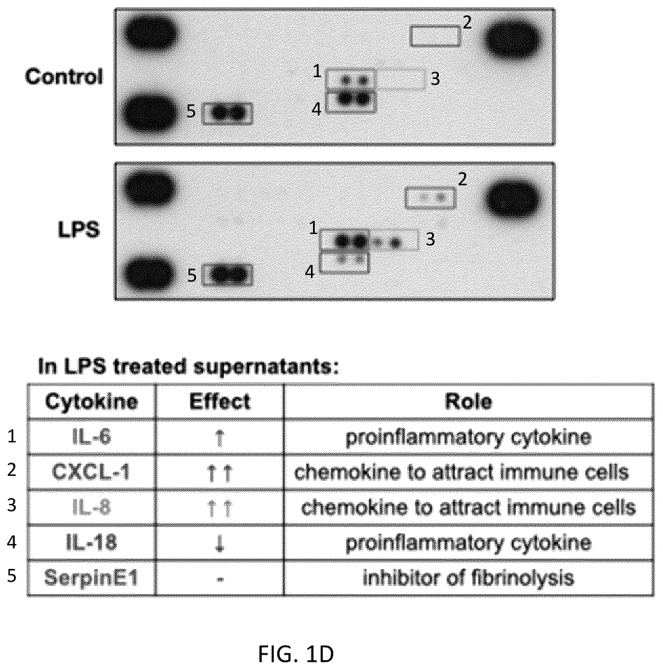

The anti-inflammatory effects of nitazoxanide are mediated via transcriptional activation of MNRR1. Nitazoxanide was identified as an MNRR1 activator from a list of 2400 FDA-approved drugs and well-characterized small molecules and natural products (FIG. 1A). By using orthogonal assays with HTR8 cells, it was found that the half-maximal effective concentration (EC₅₀) of nitazoxanide is 0.115 μM (FIG. 1B). To evaluate whether the anti-inflammatory effects of this compound are mediated via activation of MNRR1, the MNRR1 dependence of the ability of nitazoxanide to mitigate the LPS-induced inflammatory response was tested in HTR8 cells. It was found that activation of TNF transcription, a previously described effect of LPS exposure in HTR8 cells (Purandare et al., iScience, 25: 105342, 2022), was reduced to control levels in an MNRR1-dependent fashion (FIG. 1C). There was also a basal increase in TNF transcription after MNRR1 knockout that was restored to control levels upon reintroduction of MNRR1. Since the placental cells need to communicate this signal to activate an immune cell response, the cytokine profile of HTR8/SVneo cells was analyzed. A cytokine array to identify differences in control and LPS-treated cells was used. It was found that the levels of the pro-inflammatory cytokine IL-6 are increased (FIG. 1D) as also observed by other studies (Fan et al., Front. Physiol., 10:1030, 2019; Anton et al., Hum. Reprod., 27:61-72, 2012). It was also identified that two important chemokines that can attract immune cells, IL-8 (Moore et al., J. Immunol., 202:3-4, 2019) and CXCL1 (Filippo et al., Blood, 121:4930-4937, 2013), are increased (FIG. 1D). Such findings may represent a novel mechanism by which placental cells can secrete immune modulators to attract immune cells.

### Example 2—Activation of MNRR1 Using Nitazoxanide Prevents Preterm Birth In Vivo

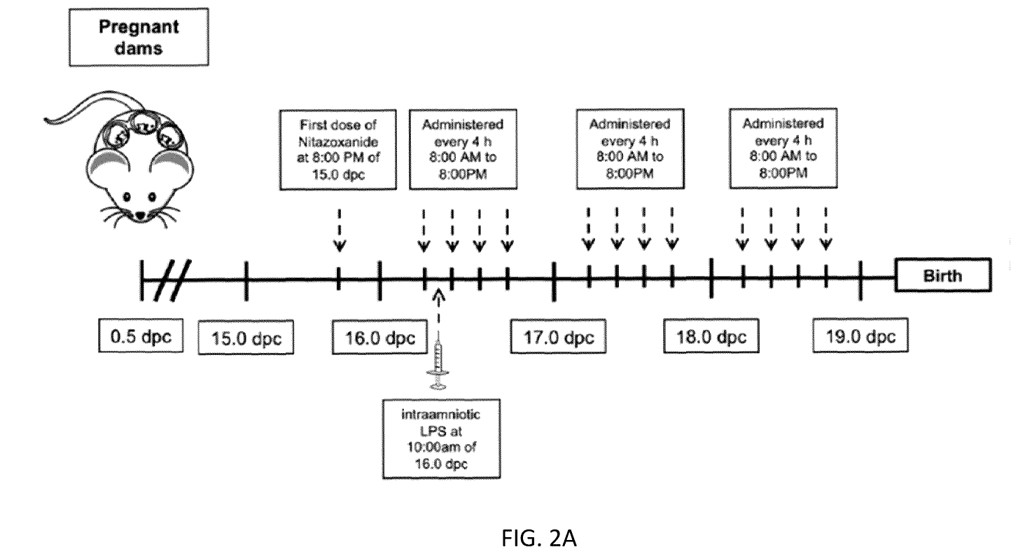

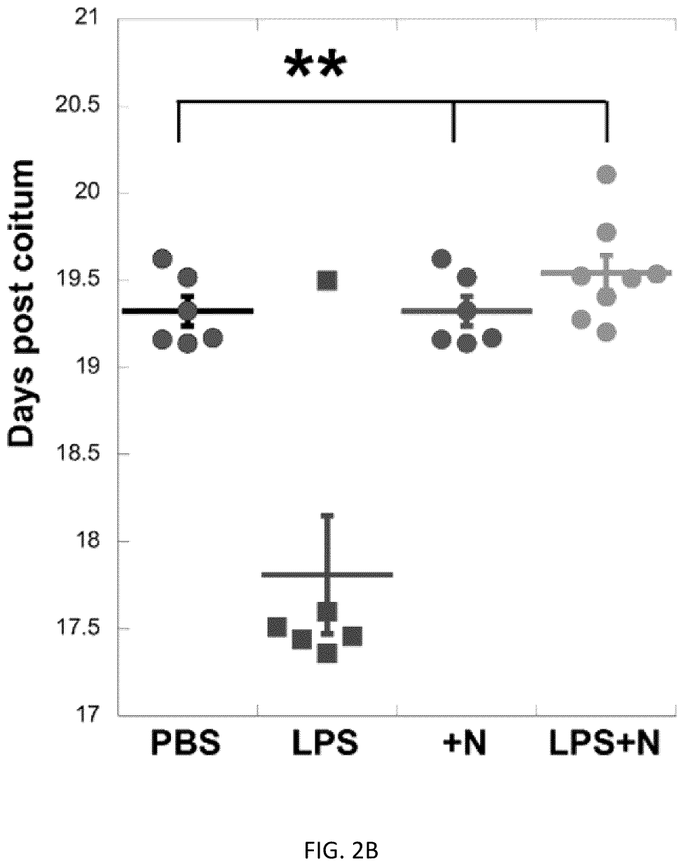

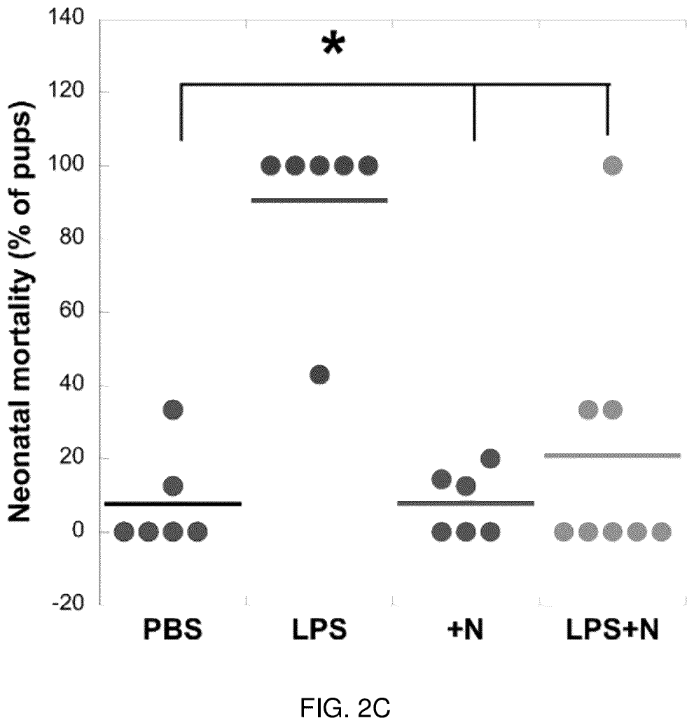

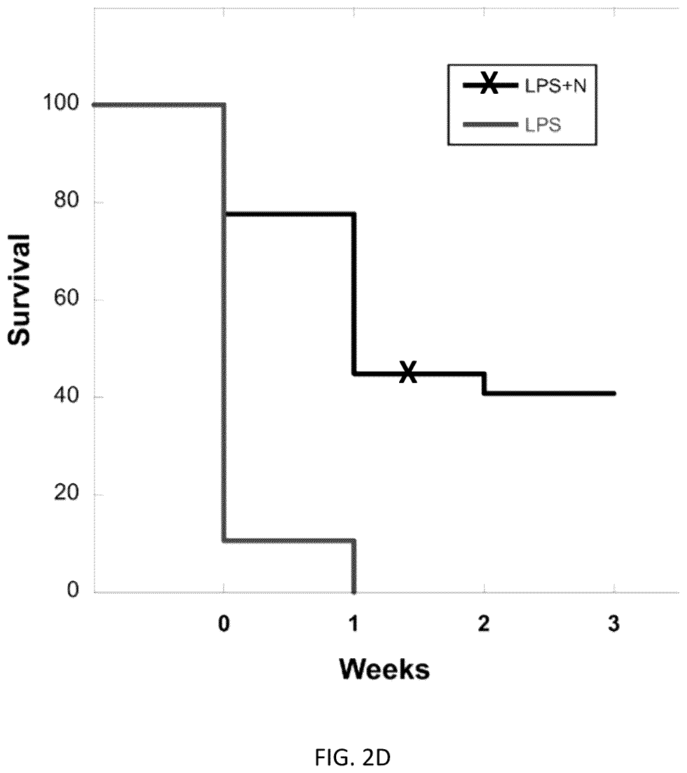

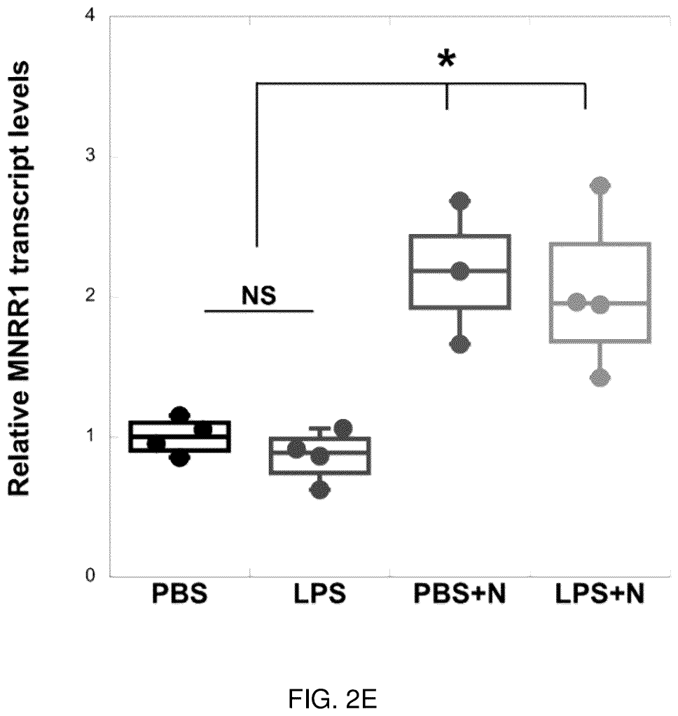

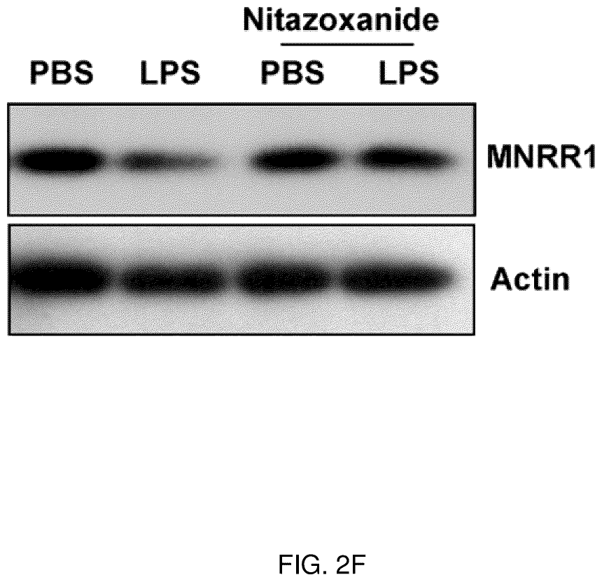

The effects of nitazoxanide were tested in a well-characterized murine model of intra-amniotic LPS injection that results in inflammation and preterm birth (Gomez-Lopez et al., J. Matern. Fetal Neonatal Med., 31:439-446, 2018; Faro et al., Biol. Reprod., 100:1290-1305, 2019; Garcia-Flores et al., Front. Immunol., 9:1291, 2018; Motomura et al., JCI Insight, 7, 2022). Dams received nitazoxanide orally and ultrasound-guided intra-amniotic injection of LPS as shown in FIG. 2A.

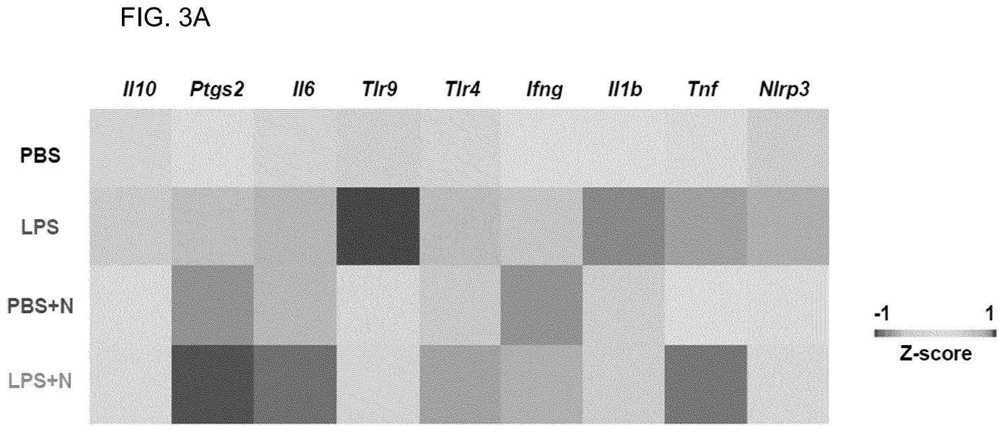

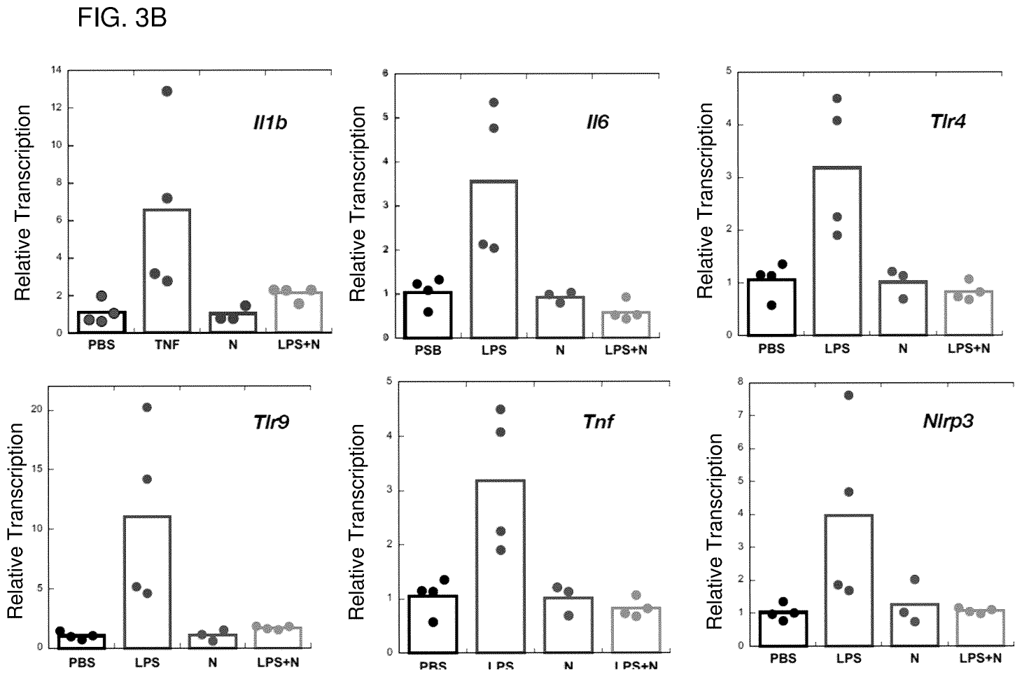

Nitazoxanide extended the gestational age, preventing LPS-induced preterm birth (delivery <18.5 days post coitum (dpc)) in all pregnant dams (FIG. 2B). Furthermore, neonatal mortality at birth was also significantly reduced in dams treated with nitazoxanide compared to controls (FIG. 2C). When neonatal mortality was assessed for up to 3 weeks, all neonates from the LPS-injected dams died within the first week of life, whereas almost one-half of the neonates born to dams treated with nitazoxanide survived up to 3 weeks (FIG. 2D). Finally, a separate group of mice was used to show that nitazoxanide treatment restored the LPS-induced reduction of MNRR1 at the transcript level in the decidua (FIG. 2E) and at the protein level in the placenta (FIG. 2F). To assess MNRR1 transcript and protein levels, separate animals were used from those that were observed for outcomes until delivery. The transcript levels of several inflammatory factors such as ll6, ll1b, Tnf, and Tlr4, among others, were assessed in the decidual samples and increased expression was found in dams intra-amniotically injected with LPS that was abrogated upon treatment with nitazoxanide (FIGS. 3A and 3B).

### Results and Discussion

It is shown here that nitazoxanide, a clinically approved drug sold commercially as Alinia® for instance, can abrogate preterm birth in an LPS-based mouse model of intra-amniotic inflammation by stimulating the transcription of MNRR1. It was previously shown that LPS treatment produces an inflammatory phenotype in a trophoblast cell line that includes reduced mitochondrial respiration and increased production of ROS and inflammatory cytokines such as TNF (Purandare et al., iScience, 25: 105342, 2022). This phenotype included reduced expression of the mitochondrial regulator MNRR1, and stimulation of this factor, either genetically or pharmacologically, with nitazoxanide reversed the inflammatory profile. Consistently, it is shown here that the anti-inflammatory effects of nitazoxanide in pregnant mice are also mediated through transcriptional activation of MNRR1 since there is no nitazoxanide-mediated benefit in MNRR1 knockout cells (FIG. 1C). There was also a basal increase in TNF transcription after MNRR1 knockout that was restored to control levels upon reintroduction of MNRR1.

Nitazoxanide is currently used as a treatment for several infectious organisms including protozoa, helminths, bacteria, and viruses (Shakya et al., Curr. Drug Discov. Technol., 15:201-213, 2018; Al-Kuraishy et al., Mol. Biol. Rep., 49:11169-11176, 2022). The molecular target of nitazoxanide is known in protozoa (Hoffman et al., Antimicrob. Agents Chemother., 51:868-876, 2007) but not in humans. Inflammation-induced preterm birth leads to significant adverse effects on maternal and fetal health (Gomez-Lopez et al., Reproduction, 164: R11-R45, 2022). Administration of nitazoxanide in pregnant mice injected with LPS prevented preterm birth and reduced neonatal mortality up to 3 weeks. It also significantly reversed the inflammation induced by LPS in these animals. Hence, this clinically approved drug appears to be worthy of further evaluation for potential use in high-risk pregnancies that may result in preterm birth.

Specifically, although recent studies have shown that intra-amniotic infection can be treated with appropriate antibiotics (Yoon et al., Am. J. Obstet. Gynecol., 221:142 e141-e142 e122, 2019; Oh et al., Am. J. Obstet. Gynecol., 221:140 e141-e140 e118, 2019; Lee et al., J. Matern. Fetal Neonatal Med., 29:2727-2737, 2016), sterile intra-amniotic inflammation currently lacks treatment (Gomez-Lopez et al., Reproduction, 164: R11-R45, 2022), thus nitazoxanide could potentially serve as a therapeutic strategy for this condition.

MNRR1 is known to function in mitochondria and in the nucleus (Aras et al., Nucleic Acids Res., 41:2255-2266, 2013; Aras et al., Mitochondrion, 20:43-51, 2015; Purandare et al., J. Biol. Chem., 293:6517-6529, 2018; Aras et al., Biochim. Biophys. Acta Mol. Cell Res., 1864:440-448, 2017; Grossman et al., Oxid. Med. Cell. Longev., 2017: 6739236, 2017), and, in a recent cellular model, its nuclear function alone as a transcriptional activator was sufficient to dampen LPS-induced inflammation (Purandare et al., iScience, 25: 105342, 2022). However, in animals, a more detailed understanding of its action in activating multiple homeostatic mechanisms (Aras et al., Proc. Natl. Acad. Sci. U.S.A, 117:32056-32065, 2020) may well uncover additional genes of interest as well as its potential utility for treating other inflammatory conditions.

### Example 3—Prevention of Infection-Induced Preterm Birth In Vivo

Using the E. coli-induced placental inflammation model and methods similar to those described in Spencer et al. (PLoS One, 16(12): e0260370, 2021 doi: 10.1371/journal.pone.0260370), nitazoxanide therapy will be tested using the same dosage as above and in Purandare et al. (Placenta 140:66-71, 8 Jul. 2023; Purandare et al., bioRxiv, Feb. 1, 2023). It is predicted that nitazoxanide will be effective in reducing or preventing pre-term birth in this test.

### Example 4—Clinical Use of Nitazoxanide

Based on the teachings provided herein, the clinical use of nitazoxanide to reduce or prevent pre-term birth is enabled.

As Nitazoxanide is a schedule b drug, it is expected to be safe. Additionally, it can be given orally. Currently, vaginal progesterone is recommended for singleton pregnancy with risk of preterm delivery (Obstet Gynecol 138(2): e65-e90, 2021; doi: 10.1097/AOG.0000000000004479). Using the discovery described herein, clinicians can use Nitazoxanide with or without progesterone, for instance using the current guidelines of the American College of Obstetrics and Gynecology. See also Shennan et al. (Int. J. Gynaecol Obstet 155(1):16-18, 2021), Lucovnik et al. (Acta Obstet Gynecol Scand. 90(10):1057-2069, 2011), Romero et al. (Semin Fetal Neonatal Med. 15(9):15-26, 2013).

### Example 5—Pseudohypoxia-Stabilized HIF2α Transcriptionally Inhibits MNRR1, a Druggable Target in MELAS

The observation that amounts of the mitochondrial regulator MNRR1 (CHCHD2, AAG10, PARK22) are reduced in several pathologies, and that restoration of its level normalizes the pathological phenotype, prompted a search for compounds that could increase MNRR1 levels. High throughput screening of a 2400-compound drug and natural products library uncovered the antifungal drug nitazoxanide and its metabolite tizoxanide as effective enhancers of MNRR1 transcription. Using the mitochondrial disease MELAS (in which various mixtures, called heteroplasmy, of wild-type and mutant mitochondrial DNA (mtDNA) coexist) as a test, we showed that treating a cybrid MELAS model with tizoxanide could restore cellular respiration, enhance mitophagy, and, importantly, shift heteroplasmy toward more wild-type mtDNA. Furthermore, in MELAS patient fibroblasts, the compound could improve mitochondrial biogenesis, enhance autophagy, and protect the fibroblasts from LPS-induced inflammation. Chemical activation of MNRR1 is thus a potential strategy to improve mitochondrial deficits seen in MELAS. Investigation of the mechanism by which MNRR1 is reduced identified that two factors compete to regulate transcription at the MNRR1 promoter—RBPJk, which stimulates it, and HIF2α, which inhibits it. In MELAS cells there is a pseudohypoxic state that stabilizes HIF2α, leading to transcriptional inhibition of MNRR1. Nitazoxanide reduces the levels of HIF2α by increasing the levels of PHD3, the prolyl hydroxylase that degrades HIF2α.

At least some of the research and results described in this Example were published as Purandare et al. (bioRxiv 2024.10.07.617011; doi: https://doi.org/10.1101/2024.10.07.617011) on Oct. 8, 2024.

Mitochondria are well known to be genetic hybrids between the products of nuclear DNA (nDNA) and mitochondrial DNA (mtDNA) [1, 2]. Mutations in mtDNA, and its multicopy nature in which thousands of copies can be present in a diploid cell, give rise to a mixture of wild-type and mutant copies, termed heteroplasmy. Although there are diseases of homoplasmy, where only mutant mtDNA is present (e.g., LHON [3]), many mtDNA diseases are heteroplasmic like MELAS (Mitochondrial Encephalomyopathy, Lactic Acidosis and Stroke-like episodes), a rare genetic disease that affects multiple organs. In such cases of a pathogenic mtDNA mutation, the higher the heteroplasmy level the more severe the phenotype [4, 5]. As a result, reducing the heteroplasmy level represents a viable treatment strategy. Doing so can involve increasing the amount of wild-type mtDNA, reducing the amount of mutant, or both.

Several pathogenic mtDNA mutations are specifically associated with the MELAS syndrome, the most common being m.3243A>G in the mitochondrial MT-TL1 gene that codes for tRNA^{Leu(UUR)}. The mutation causes hypophysiological mitochondrial protein translation and synthesis and can attain heteroplasmy levels >50% [6]. Mitochondria that cannot adequately translate and assemble electron transport chain (ETS) subunits produce insufficient energy to meet the requirements of their resident tissues along with other deficiencies, resulting in the characteristic MELAS multi-organ dysfunction whose sequelae are difficult to treat. For example, the overall energy deficiency stimulates mitochondrial proliferation in endothelial cells, precipitating the angiopathy and impaired blood perfusion that exacerbate organ damage and the stroke-like episodes. Deficiency of nitric oxide (NO), which regulates smooth muscle relaxation, contributes to these sequelae, including hypertension, fatigue, and memory loss. Treatments that increase NO levels are common clinical approaches currently under study. However, there are no specific standard treatments available to MELAS patients and many die between the ages of 10 and 35, underscoring a considerable unmet patient need.

Mitochondrial Nuclear Retrograde Regulator 1 (also called CHCHD2, PARK22, AAG10) is a biorganellar regulator of mitochondrial and nuclear function. MNRR1 was discovered in a computational screen to identify factors regulating the ˜90 proteins that comprise the oxidative phosphorylation complex [7]. Work since then has revealed that mitochondrial MNRR1 binds to cytochrome c oxidase (COX) to activate respiration [8, 9] and interacts with Bcl-xL to impede the extrinsic apoptosis cascade [10]. Nuclear located MNRR1, along with the protein RBPJk, functions to activate transcription by binding to the conserved oxygen-responsive element (ORE) of its own promoter as well as to a host of other stress-response genes [11, 12]. Recent work from our group demonstrated that ectopic expression of MNRR1 could rescue the MELAS mitochondrial phenotype in vitro by increasing OXPHOS and the expression of CREBH target genes, which led to a significant decrease in heteroplasmy [12]. We also found that the transcription of MNRR1 was inhibited in MELAS cybrid cells. Thus, we sought small molecules that could activate transcription of MNRR1 (and its target genes) to improve MELAS heteroplasmy and its associated OXPHOS pathologies. We identified nitazoxanide as an activator of MNRR1 transcription and uncovered a novel mechanism by which MNRR1 transcription is inhibited in MELAS and which is averted by the drug to enhance transcription.

### Results

### High Throughput Screen to Identify MNRR1 Activators

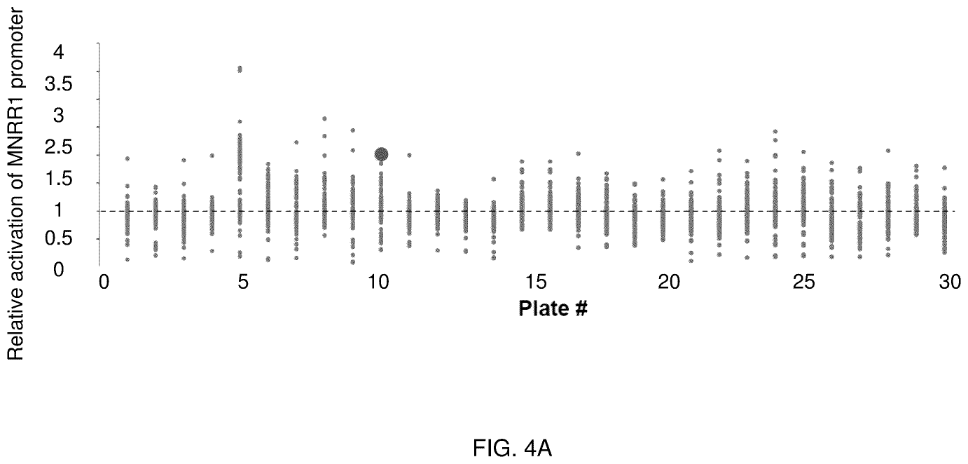

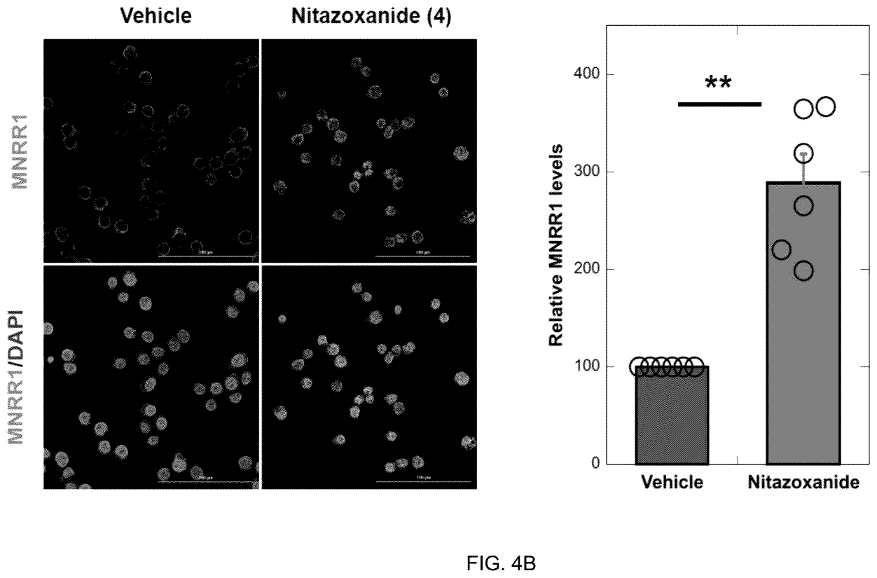

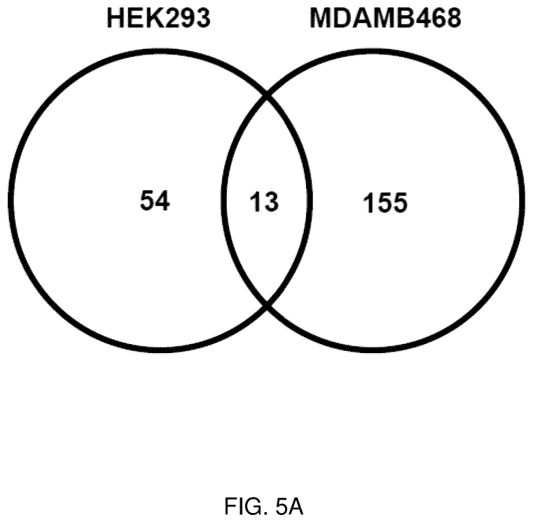

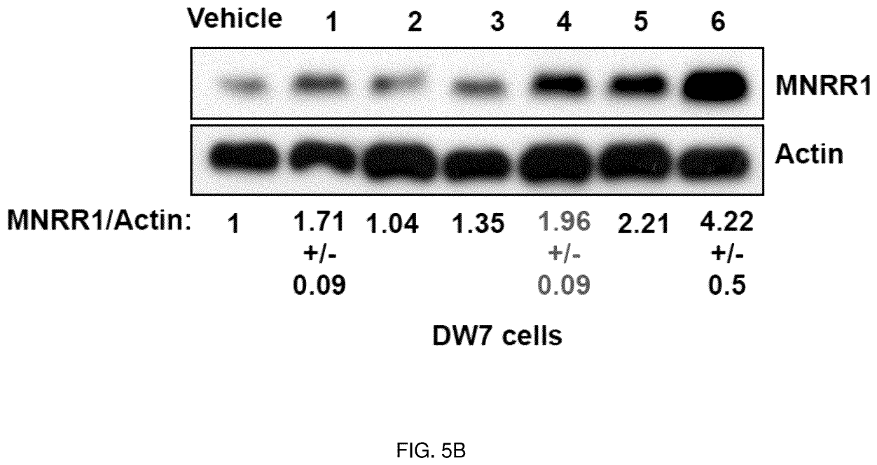

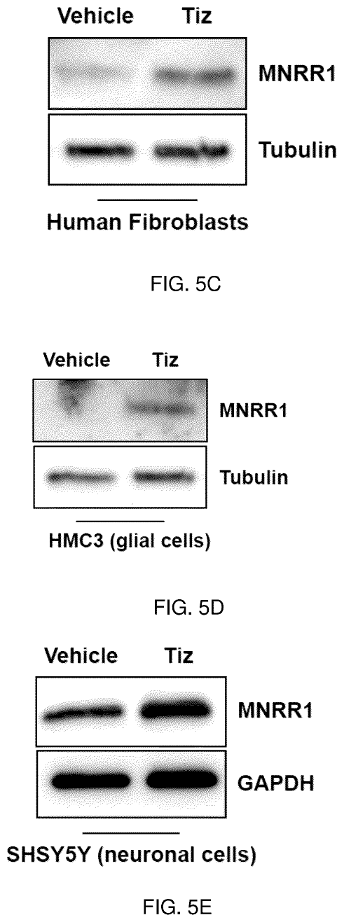

We have previously shown that activation of MNRR1 using exogenous overexpression rescues mitochondrial deficit in MELAS cybrid cells and stable overexpression was able to shift heteroplasmy towards wild-type (WT) mitochondrial DNA [12]. Hence, we were interested in identifying chemical activators of MNRR1 that could be repurposed as a therapeutic intervention in MELAS patients. To identify activators of MNRR1, a screen of 2400 FDA-approved compounds was performed using two independent cell lines, HEK293 and MDA-MB-468, stably expressing the MNRR1-promoter driven luciferase reporter. Compounds were selected that activated the reporter by at least 50% or higher (FIG. 4A), identifying 54 and 155 compounds on the HEK293 and MDA-MB-468 screens, respectively (FIG. 5A). Thirteen compounds were common to both cell lines and, of these, 7 were selected based on their lower toxicity profile. From these, six were selected based on clinical availability. We then validated these compounds in a 143B osteosarcoma cell line (DW7) cybrid with ˜73% MELAS mutant mtDNA (m.3243A>G) in which MNRR1 levels are reduced [12](FIG. 5B). We chose compound 4 since this was available as a clinical formulation that has been used for in vivo testing and shown to increase MNRR1 transcripts and protein levels [13]. Also confirmed was the increase of MNRR1 in one of the original cell lines used for identifying the activators—MDAMB468 (FIG. 4B) as well as in several other human cell lines (using tizoxanide, the active nitazoxanide metabolite—see below) (FIGS. 5C-5E).

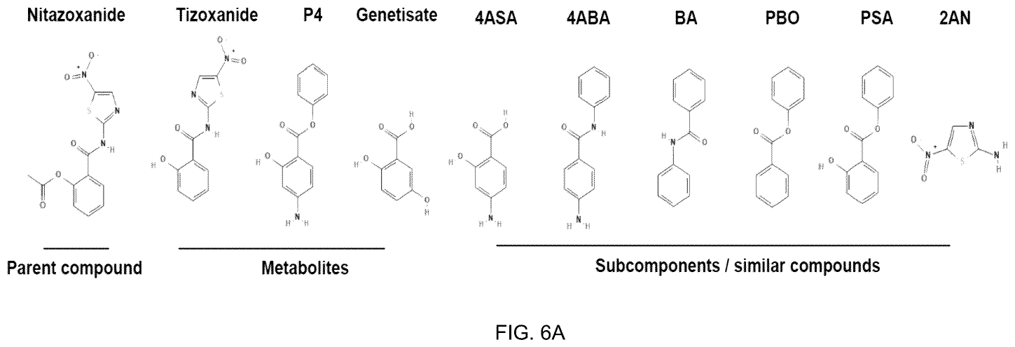

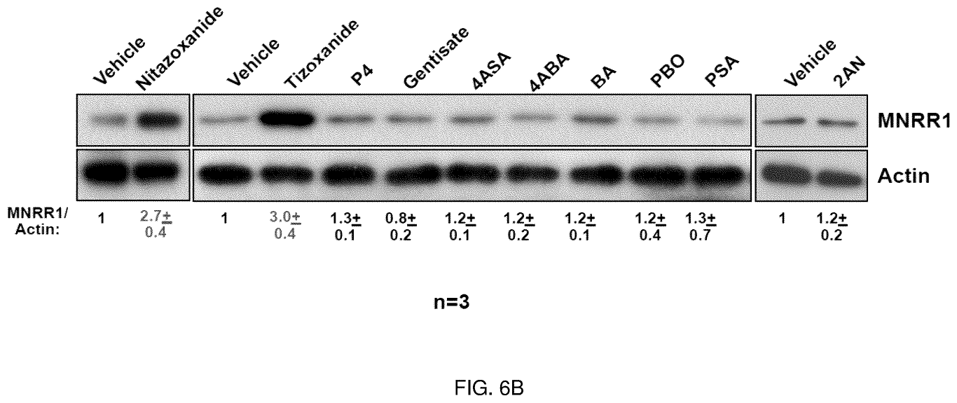

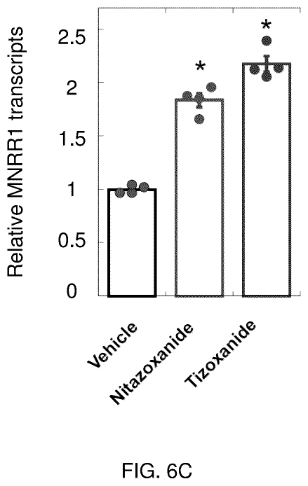

Nitazoxanide in cells is metabolized to tizoxanide (>97%) plus minor metabolites such as aminonitrothiazole and gentisate [14]. Only nitazoxanide- and tizoxanide-treated DW7 cells displayed a significant increase in the protein levels of MNRR1 (FIG. 6A). To confirm that the effects were at the transcriptional level, we measured MNRR1 transcripts and observed that both compounds induced its transcription (FIG. 6B).

### MNRR1 Activation Using Tizoxanide Enhances Mitochondrial Biogenesis and Mitophagy to Shift Heteroplasmy in MELAS Cybrid Cells

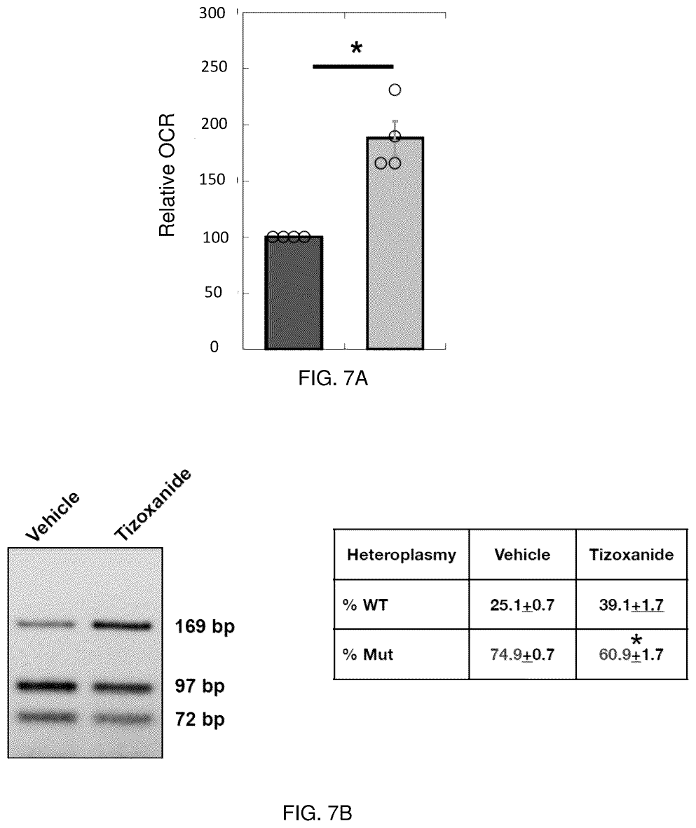

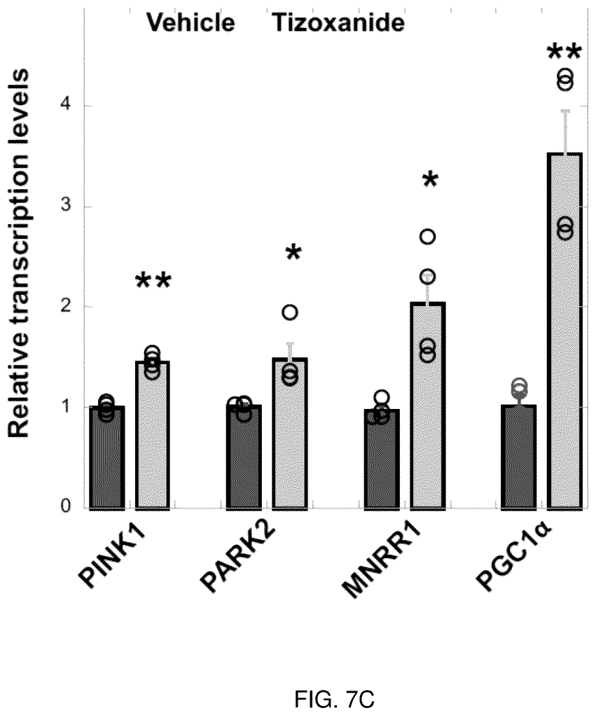

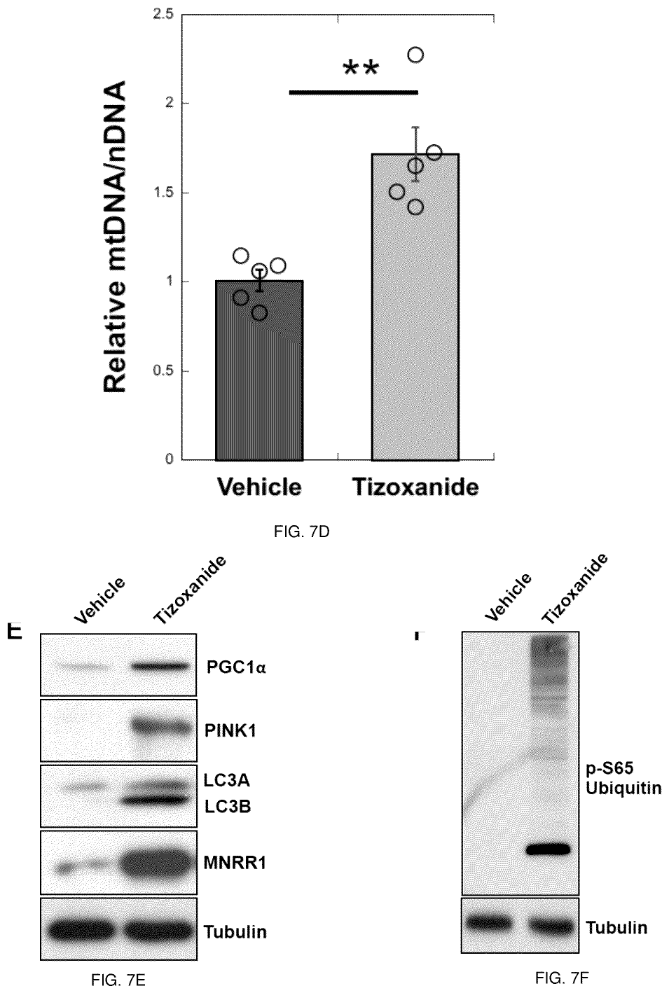

We first confirmed the effects of MNRR1 activation by measuring oxygen consumption, which was increased (FIG. 7A). We had previously shown that MNRR1 overexpression induces homeostatic pathways such as mitophagy and mitochondrial biogenesis to aid in rescuing the phenotype. We had also shown that MNRR1 overexpressing cells display a reduction in heteroplasmy, making it an attractive therapeutic target for MELAS [12]. We therefore tested tizoxanide on MELAS cybrid cells and found it increased the proportion of WT mtDNA, by about 14% here, as shown by Haelll digestion of the mtDNA fragment harboring the MELAS point mutation (FIG. 7B). Stable overexpression of MNRR1 in MELAS cybrid cells activated multiple homeostatic genes (FIG. 7C) and restored healthy mitochondria, presumably in part by stimulating mtDNA synthesis (FIG. 7D) by increasing PGC1α (FIGS. 7C, 7E) and enhancing mitophagy. Increased mitophagy is shown by increase of PINK1 (FIGS. 7C, 7E), autophagosomal proteins LC3A and LC3B (FIG. 7E), and by increased levels of the mitophagy marker pSer-65 ubiquitin (FIG. 7F) [15]. Taken together, these results suggest that tizoxanide increases MNRR1 levels and thereby induces the downstream pathways that rescue defective mitochondrial function in MELAS cybrid cells.

MNRR1 Activation Using Tizoxanide in MELAS Patient Fibroblasts Enhances Mitochondrial Function and Mitophagy and Protects from LPS-Induced Inflammation

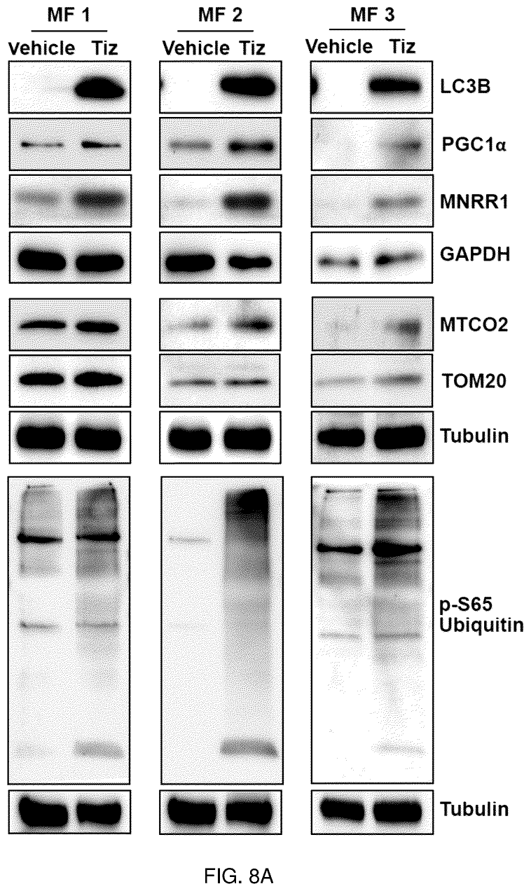

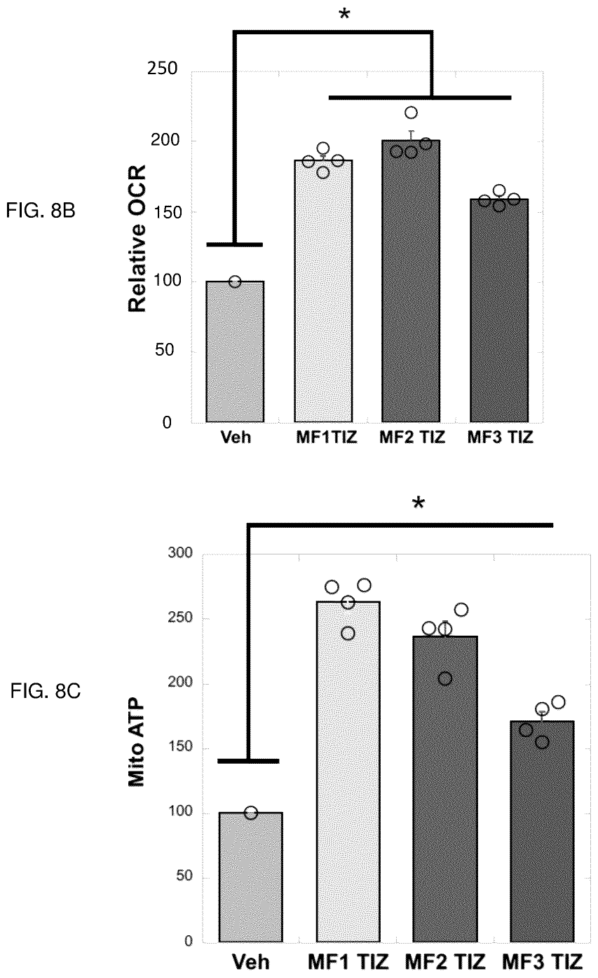

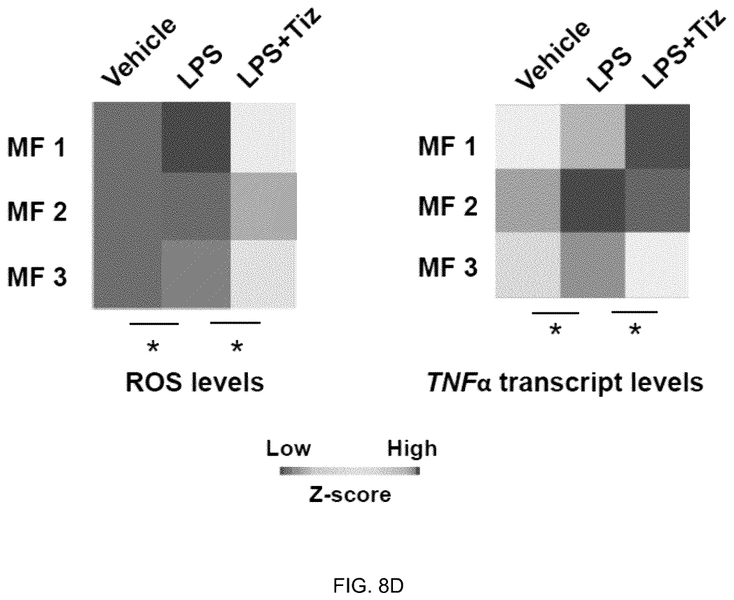

In primary fibroblasts from three independent MELAS patients, we found that activation of MNRR1 enhances mitophagy (as seen via LCB and pSer-65 ubiquitin levels) and mitochondrial biogenesis (PGC1α, MTCO2, TOM20 levels) (FIG. 8A). Furthermore, we found that OCR (FIG. 8B) and mitochondrial ATP (FIG. 8C) levels were enhanced. Since we recently found in a cell culture model that activation of MNRR1 resolved the effect of LPS-induced inflammation [16], and that mitochondrial diseases are associated with a pro-inflammatory phenotype [17], we also asked if we could rescue the effects of LPS in these patient fibroblasts. We found that LPS induces a pro-inflammatory response as judged by increased ROS and TNF levels, and this response can be blocked by tizoxanide (FIG. 8D), suggesting that MNRR1 activation is protective in these cells.

### Nitazoxanide Acts by Reducing HIF2α Protein Levels in MELAS Cybrid Cells

Nitazoxanide was identified by screening a library for transcriptional activators of MNRR1 using 952-bp promoter luciferase-expressing stable cell lines. We therefore sought to identify the region on the MNRR1 promoter that responds to nitazoxanide with MNRR1 induction. To this end we generated 200-bp deletions in the MNRR1 promoter and cloned them into the pGL4-basic luciferase vector (FIG. 9A). Upon testing the responsiveness of each of these constructs to nitazoxanide and tizoxanide in MELAS cybrid cells, we observed that a deletion of the 801-952 region on the promoter (A801-952) failed to display activation (FIG. 9B), suggesting that this promoter region was affected by nitazoxanide. Bioinformatic analysis of this region identified six bona fide binding sites for transcription factors (TFs)—Zeb1, HIF, ZFX, SMARCA3, ZNF35, and RBPJk (FIG. 9C). RBPJk binds to the core 13-bp element that we previously characterized to be responsive to moderate hypoxia and labeled as the oxygen responsive element (ORE) [11]. Of the six TFs, Picard et al. [6], who initially characterized the cells, identified only HIF2α to be transcriptionally induced (FIG. 9D). We assessed the level of both and found that HIF1α was not increased in the MELAS cells whereas HIF2α levels were higher in heteroplasmic MELAS cybrid cells (DW7) (FIG. 9E) as compared to the control cybrids (CL9) [6, 18]. To test whether tizoxanide was acting through HIF2α, we measured its protein levels and found that HIF2α was inversely proportional in a concentration dependent manner to MNRR1 in MELAS cybrid cells treated with tizoxanide (FIG. 10A). To test whether HIF2α is acting specifically via the hypoxia response element (HRE), we generated a deletion of the HRE in the MNRR1 promoter (FIG. 10C, right bar). To our surprise, we found that the deletion of the HRE could not reverse the inhibition in MELAS cells, whereas deletion of the ORE could rescue the effects (FIG. 10B). To evaluate this confounding effect, we tested an HRE-harboring reporter in the MELAS cybrid cells (DW7) and found it to be more active than in the control cybrid cells (CL9) (FIG. 10C). In the same control cybrid cells, we could also overexpress HIF2α and repress MNRR1 transcription (FIG. 10D). However, since this effect was not through the HRE in the MNRR1 promoter (FIG. 10B), we again examined the sequence of the 800-952 region on the promoter and uncovered a second HRE in the reverse orientation on the opposite strand of the ORE where RBPJk binds (FIG. 10E), thus providing a possible explanation for the effects seen in FIG. 10B. We previously showed that MNRR1 forms a required transcriptional complex with RBPJk at the ORE and that constitutively active RBPJk can bypass the need for MNRR1 to activate transcription [11].

### RBPJk and HIF2α Compete for Binding at the ORE in the MNRR1 Promoter to Regulate Transcription

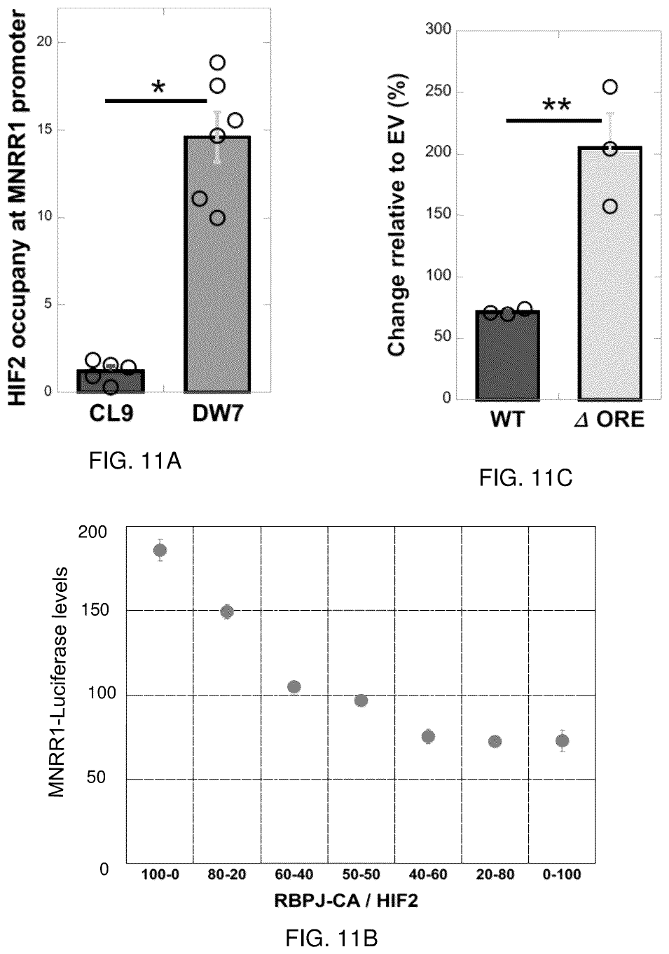

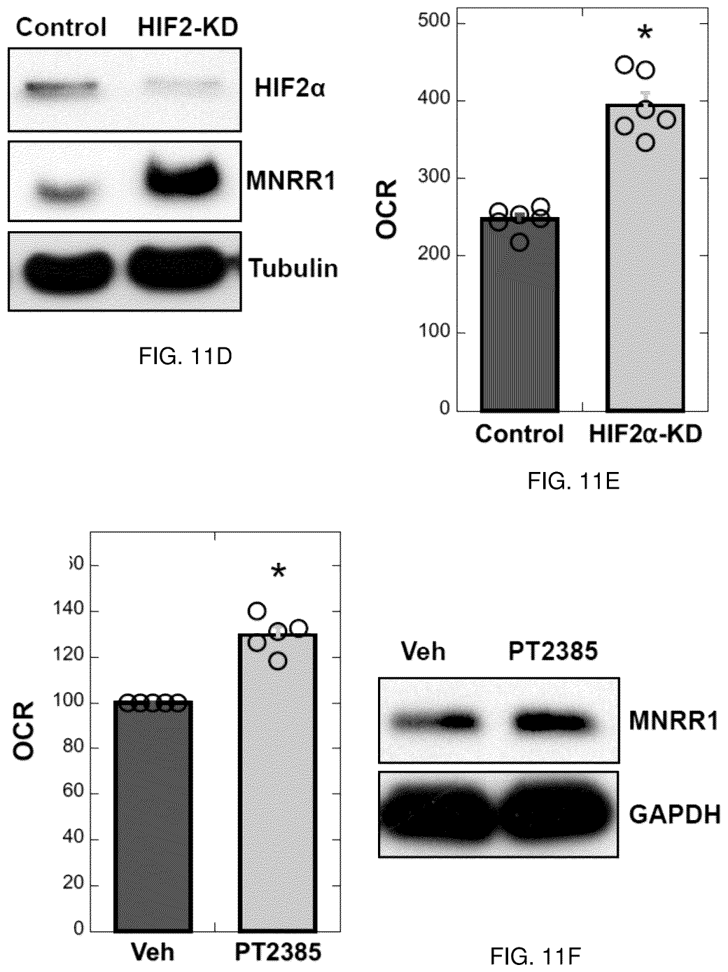

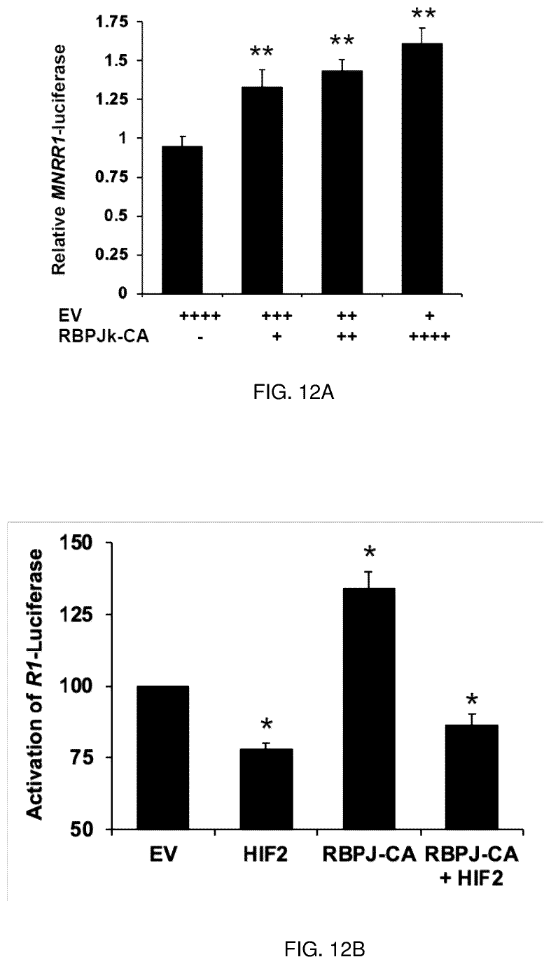

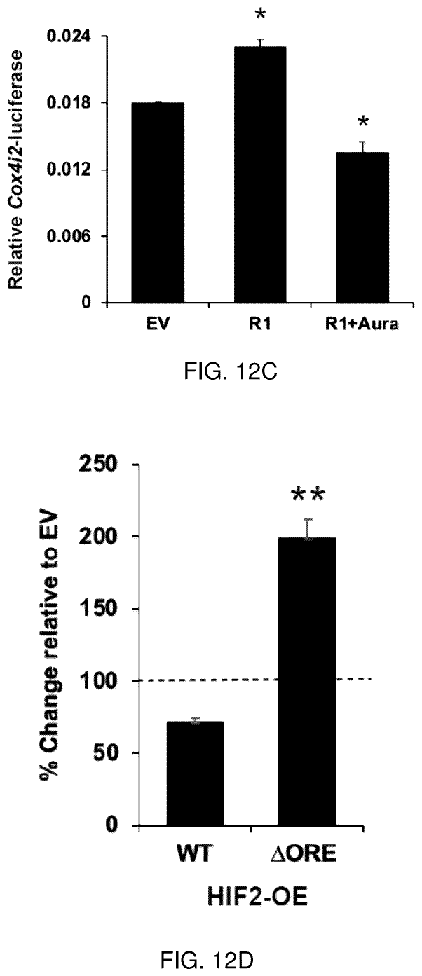

To dissect the effect of HIF2α and RBPJk at the ORE, we first confirmed by chromatin immunoprecipitation that HIF2α can bind at the MNRR1 promoter (FIG. 11A). Using a constitutively active version of RBPJk (CA-RBPJk), we could rescue defective transcription of MNRR1 in MELAS cybrid cells (FIG. 12A) and block these effects by overexpression of HIF2 (FIG. 12B). We also titrated RBPJk and HIF2α in MELAS cybrid cells and found that HIF2α can compete with, and inhibit, transcription induced by RBPJk (FIG. 11B). Furthermore, a chemical inhibitor that blocks binding of RBPJk to DNA (Auranonfin) was able to block the effects of MNRR1 induced transcription (FIG. 12C) whereas the effects of HIF2α overexpression were blocked by deletion of the ORE (FIG. 11C). Taken together with a previously published report that HIF interacts with RBPJk to inhibit transcription [19], we hypothesize that nitazoxanide relieves the inhibitory effect of HIF2α atthe ORE to facilitate transcriptional activation via RBPJk. At the protein level, depletion of HIF2α in the MELAS cybrid cells increases MNRR1 (FIG. 11D) and oxygen consumption (FIG. 11E). As a further confirmation, we specifically inhibited HIF2α with the compound PT2385 [20] and observed a rebound increase in both OCR and MNRR1 protein in MELAS cybrid cells (FIG. 11F). Consequently, we propose that HIF2α acts in two ways to regulate MNRR1 transcription: (1) it binds to the HIF site shown at positions 88-92 of SEQ ID NO: 25 (FIG. 9C) in the 800-952 bp region of the in the MNRR1 promoter to activate transcription, and (2) it binds on the complementary stand of the ORE site shown in orange (FIG. 9C) to inhibit transcription. A balance between these mechanisms is imposed by the ratio of RBPJk to HIF, as is suggested in FIG. 11B.

### PHD3 Levels are Reduced in MELAS Cybrid Cells and Enhanced by Nitazoxanide to Increase MNRR1 Levels

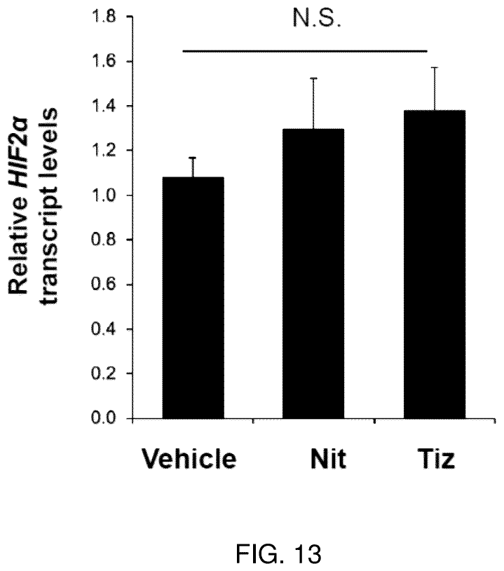

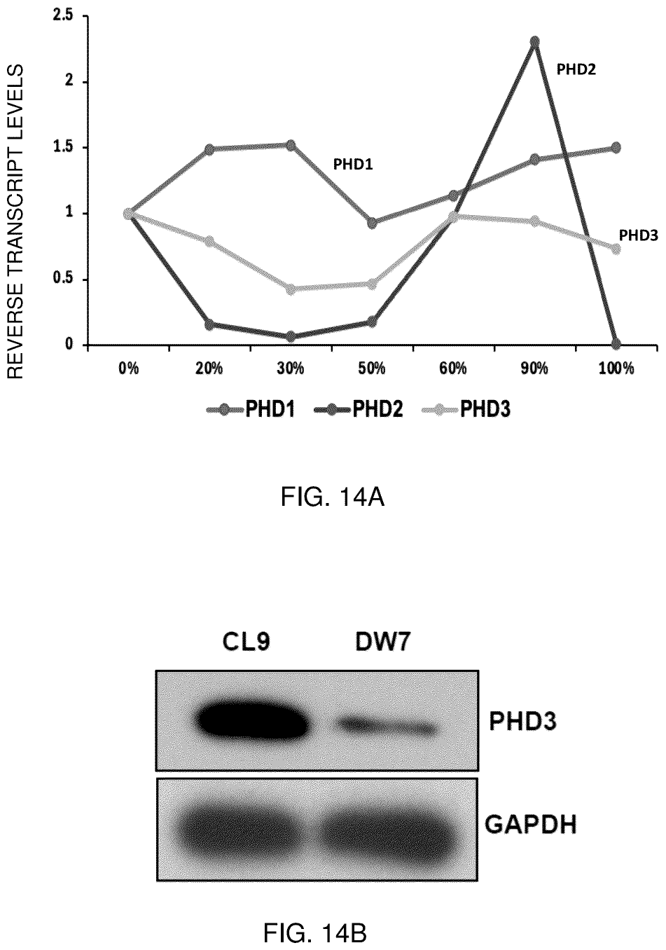

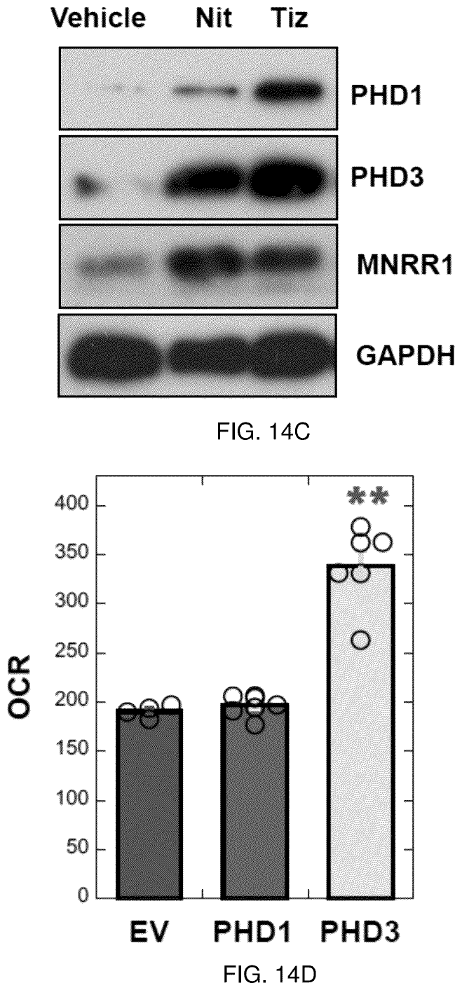

The absence of any change in HIF2α transcript levels with nitazoxanide and tizoxanide (FIG. 13) suggested that HIF was showing greater protein stability. Hence, we assessed the levels of all three prolyl hydroxylases (PHD1, 2, and 3) in the MELAS cybrid cells using published transcriptomics data (FIG. 14A) [6]. Of these, we found that only PHD2, which shows specificity for HIF1α, was increased at high heteroplasmy, consistent with the reduction of HIF1α in DW7 versus CL9 cells (FIG. 9E). Next, we assessed the levels of PHD3 and found that it was reduced in the DW7 MELAS cells (FIG. 14B). Since HIF2α is increased in DW7 cells (FIG. 9E), we examined the role of PHD1 and PHD3. We found that PHD3 is most increased by tizoxanide at the protein level (FIG. 14C) and, when overexpressed, uniquely stimulates respiration in DW7 cells (FIG. 14D), suggesting that PHD3 may be the prolyl hydroxylase responsible for targeting HIF2α. PHD2 was not evaluated here as studies have shown this enzyme to be selective for HIF1 [21, 22], whose levels in MELAS cells were not correlated with heteroplasmy [6]. These results suggest that nitazoxanide and tizoxanide transcriptionally induce MNRR1 by reducing the inhibitory effect of HIF2α and facilitating its activation via RBPJk.

### DISCUSSION

This researches showed that MELAS cells contain a reduced amount of MNRR1 and that genetically restoring the amount alleviated much of the pathophysiological phenotype such as reduced energy generation and increased ROS [12]. This suggested that activation of MNRR1 could improve mitochondrial deficits associated with MELAS and stimulated a search for a small molecule that would restore MNRR1 levels. The MNRR1 transcriptional activator nitazoxanide was discovered by high throughput screening of a drug and natural products chemical library. Nitazoxanide and its metabolic breakdown product tizoxanide can restore expression and thereby function in MELAS cybrid cells and, importantly, in primary fibroblasts from MELAS patients. In seeking to identify the drug's mechanism of action, it has been discovered that MELAS cells contain increased amounts of HIF2 at normoxia, and that HIF2 binds at the MNRR1 promoter to inhibit transcription.

In parasites, nitazoxanide inhibits pyruvate:ferredoxin oxidoreductase (PFOR), a key enzyme utilized by anaerobes in the oxidative decarboxylation of pyruvate to acetyl-CoA and CO₂, whereas in humans nitazoxanide has no known target. We first identified the region of the MNRR1 promoter where nitazoxanide acts and then narrowed to the specific transcription factors—HIF2α and RBPJk—that bind to overlapping regions in the ORE part of the promoter to regulate transcription (FIG. 10E). We have previously shown that RBPJk binds to and mediates transcription at the ORE [11] and now identify a second factor—HIF2α—that can bind to the MNRR1 promoter (FIG. 10E) to regulate transcription. These factors compete with each other: active RBPJk can enhance transcription of MNRR1 and HIF2α competes with and represses this effect. In MELAS cybrid cells, which show a pseudohypoxic state, expressing higher levels of HIF2α, MNRR1 is transcriptionally inhibited. With regard to the action of nitazoxanide, we propose a novel mechanism of post-translational regulation: we found that PHD3, the prolyl hydroxylase that is largely responsible for targeting HIF2α for degradation by the ubiquitin proteasome [23, 24], is upregulated by tizoxanide, thereby increasing MNRR1 levels. Since the anti-inflammatory effect of nitazoxanide was explicitly shown to depend on MNRR1 [13], this effect of nitazoxanide will be an important focus of future studies.

The patient fibroblasts do not show a shift of heteroplasmy after tizoxanide treatment, unlike the shift toward wild-type seen in 70% MELAS cybrid cells (FIG. 7B). This may result from the low heteroplasmy levels (<50%) in the patient fibroblasts, consistent with distinct nuclear responses for each heteroplasmy range previously seen in the cybrid model of MELAS [6]. Lower heteroplasmy levels may not induce sufficient stress in these glucose-grown cells to alter heteroplasmy.

Nitazoxanide is the prodrug formulation of tizoxanide, a commonly used FDA-approved antiprotozoal effective against diarrheal symptoms caused by Giardia or Cryptosporidium. Nitazoxanide is deacetylated in vivo to tizoxanide, which has antioxidant properties and is a known inhibitor of iNOS [25]. Both properties are likely major contributors for the observed improvements in lung and other organ damage in SARS-Cov-2 patients [26]. Consistently, MNRR1 reduction increases ROS production [9]. Thus, nitazoxanide has the potential to also alleviate MELAS beyond activation of MNRR1 transcription. Furthermore, this recently rekindled interest in the broad spectrum of tizoxanide applications to human health has led to new inquiry. The generation of more plasma-stable congeners that replace the acetyl group substituent attached to the hydroxyl group with a more stable formamyl group [27], as well as novel approaches for tizoxanide quantification in vitro and in vivo, have recently been published [28, 29]. Further studies will elucidate how increased plasma concentrations and systemic exposure to the active tizoxanide moiety will be tolerated and distributed in in vivo models of infection, cancer, and other maladies with unmet needs.

In summary, we have identified nitazoxanide and its metabolite tizoxanide as a drug that stimulates MNRR1 transcription and shown that it reduces heteroplasmy in a cell culture model of MELAS and improves the phenotype in MELAS patient fibroblasts. Given its established safety profile, it could be usefully evaluated for diseases like MELAS and other inflammatory conditions wherein MNRR1 levels become reduced [16]. The observation that activation of MNRR1 can protect from inflammatory stress is crucial since patients with MELAS and other mitochondrial diseases have a higher susceptibility to infections and bacterial sepsis [17]. Hence, these results are also consistent with our recent findings that activation of MNRR1 can prevent inflammation induced preterm birth in vivo [13]. Furthermore, we have identified increased HIF2 at normoxia as the cause of reduced MNRR1 in MELAS cells, uncovering a potential new therapeutic target. By contrast, most current studies target symptomatic relief such as by increasing nitric oxide levels to ameliorate episodic stroke-like episodes [e.g. 26].

### Materials and Methods

Cell lines: The human embryonic kidney cell line HEK293, the triple-negative breast cancer cell line MDA-MB-468, the human first trimester placental cells HTR8/SVNeo (HTR), SHSY5Y cells, and HMC3 were obtained from the ATCC (Manassas, VA). The MELAS cells (143B human osteosarcoma cybrid) were a kind gift from Dr. Douglas Wallace. The HEK293 and human fibroblast cells were cultured in DMEM with L-glutamine and D-Glucose (Gibco, Billings, MO) supplemented with penicillin-streptomycin (HyClone, Logan, MT) and 10% fetal bovine serum (FBS) (Sigma Aldrich, St. Louis, MO); the MDA-MB-468 cells were cultured as above but plus 1 mM pyruvate; the HTR were cultured in Roswell Park Memorial Institute Medium (RPMI) (HyClone) supplemented with 5% FBS plus penicillin-streptomycin; and the MELAS cells were grown in DMEM with 1 mM pyruvate supplemented with non-essential amino acids (Gibco), 50 μg/ml uridine, and 10% FBS. The SHSY5Y cells were cultured DMEM/F12 with L-glutamine and D-Glucose (Corning, Corning, NY) supplemented with Penicillin-Streptomycin (HyClone) and 10% fetal bovine serum (FBS) (Sigma Aldrich) in EMEM with the same additives.

Chemicals: Tizoxanide, nitazoxanide, and genetisate were obtained from Selleckchem (Houston, TX), Phenyl-4-aminosalicylic acid was obtained from A2Bchem (San Diego, CA), 2-Amino 5-nitrothiazole was from Sigma, and the remaining compounds (4-Aminobenzanilide, 4-Amino salicylic acid, benzanilide, phenyl benzoate, and phenyl salicylic acid) were from Santa Cruz Biotechnology (Dallas, TX). All compounds were solubilized in DMSO (used as vehicle control in all experiments with these compounds). LPS (Lipopolysaccharide from Escherichia coli 0111: B4) was purchased from Invivogen.

Plasmids: The MNRR1 promoter luciferase reporter plasmid and pRL-SV40 Renilla luciferase expression plasmids have been described previously [11]. The HRE-Luciferase plasmid was purchased from Addgene (#26731). All expression plasmids were purified using the EndoFree plasmid purification kit from Qiagen (Germantown, MD).

Cell lines, culture, and Z′ determination: Stable cell line generation: HEK293 or MDA-MB-468 cells were transfected with pGL4-MNNR1-luciferase, selected with 0.5 μg/mL (for 293) or 1 μg/mL (for 468) puromycin for 2 weeks, and subcloned by limiting dilution. MDA-MB-468-MNRR1-luc and HEK293-MNRR1-luc cells from multiple clones (5000-7500 cells) were plated in 100 μL of complete medium overnight and treated with 10 μM of each compound from the library for 24 h. The medium was aspirated from each well to a volume of 25 μL, and 25 μL Bright Glo (Promega, Madison, WI) luciferase detection reagent was added to each well 10 min prior to luminescent determination with a FlexStation 3 Multimode Microplate Reader (Molecular Devices, San Jose, CA). In the absence of a known MNRR1-inducing small molecule, we chose MNRR1 overexpression (since MNRR1 induces its own expression [9]) as a potential positive control. The Z′ factor is a value used to identify strong candidates and can be calculated using a previously described method [30]. The range of this value is negative infinity to one, with >0.5 as a very good assay, >0 an acceptable assay, and <0 an unacceptable assay. The values obtained for this screen were >0, but we were unable to identify any candidates with strong values of 0.5 to 1.

MicroSource Spectrum Collection: Despite the low Z′ values for MDA-MB-468-MNRR1-luc and HEK293-MNRR1-luc, pilot screens were performed with both using the MicroSource Spectrum Collection (Gaylordsville, CT). The Spectrum Collection is comprised of ˜2400 small molecules and natural products that are known drugs or otherwise biologically well-characterized. This library contains a manageable number of compounds in 10 mM DMSO stocks that can be tested without the need for advanced liquid handling. Additionally, the use of biologically well-characterized compounds facilitates the rapid identification of pathways and signaling networks likely to be of interest to the investigator. Dry powder stocks of the compounds that provided the most robust response in both cell lines and that did not have chemical liabilities that would preclude their use in cultured cells or, potentially, in human subjects, were then obtained from commercial sources. For HTS, cells were plated, treated, and measured for luciferase expression as described for clone identification; all HTS compounds were added to a final concentration of 10 μM and MNRR1-luciferase expression was measured after 24 h.

Since highly expressing clones that provided a Z′ value between 0.5-1.0 were elusive and there is a general lack of MNRR1 activators that could be used as positive controls, the criterion of accepting modestly enhanced transcription (1.8-2.8-fold) in each of two cell lines was used. Thus, a pilot screen with the MicroSource Spectrum Collection was performed in MNRR1-luciferase clones from MDA-MB-468-MNRR1-luc and HEK-293-MNRR1-luc cells. Compounds enhancing transcription in both cell lines were considered for further scrutiny with orthogonal assays to evaluate MNRR1 gene and protein expression. Nitazoxanide emerged as a validated MNRR1 activator and was studied further using in vitro assays in MELAS cell lines and primary fibroblasts to determine whether chemically induced MNRR1 expression could improve known human pathologic mitochondrial deficiencies. In addition to the lack of a known positive control, we hypothesize that the low Z′ values observed with our MNRR1-luciferase cell lines were due to the relatively low levels of MNRR1 expressed at baseline in monolayer cultures of MDA-MB-468 and HEK293.

Transient transfection of MELAS cells: MELAS cells were transfected with the indicated plasmids using TransFast transfection reagent (Promega) according to the manufacturer's protocol. A TransFast:DNA ratio of 3:1 in serum and antibiotic free medium was used. Following incubation at room temperature for ˜15 min, the cells were overlaid with the mixture. The plates were incubated for 1 h at 37° C. followed by replacement with complete medium and further incubation for the indicated time.

Real-time polymerase chain reaction (RT-PCR): Total cellular RNA was extracted from MELAS cells with a RNeasy Plus Mini Kit (Qiagen) according to the manufacturer's instructions. Complementary DNA (cDNA) was generated by reverse transcriptase polymerase chain reaction (PCR) using the ProtoScript® II First Strand cDNA Synthesis Kit (New England Biolabs, Ipswich, MA). Transcript levels were measured by real time PCR using SYBR green on an ABI 7500 system. Real-time analysis was performed by the ΔΔ^{Ct} method [31]. The primers used were MNRR1 forward: 5′-CACACATGGGTCACGCCATTACT-3′ (SEQ ID NO: 9), reverse: 5′-TTCTGGGCACACTCCAGAAACTGT-3′ (SEQ ID NO: 10); 18s forward: 5′-CCAGTAAGTGCGGGTCATAA-3′ (SEQ ID NO: 11), reverse: 5′-GGCCTCACTAAACCATCCAA-3′ (SEQ ID NO: 12), PHD1(EGLN2) forward: 5′-ACATCGAGCCACTCTTTGAC-3′ (SEQ ID NO: 13), reverse: 3′-TCCTTGGCATCAAAATACC-5′ [32](SEQ ID NO: 14); PHD3(EGLN3) forward 5′-TCAAGGAGAGGTCTAAGGCAA-3′ (SEQ ID NO: 15), reverse: 3′-ATGCAGGTGATGCAGCGA-5′ [33](SEQ ID NO: 16) and HIF2α (EPAS1) forward: 5′-CACCAAGGGTCAGGTAGTAA-3′ (SEQ ID NO: 17), reverse: 3′-AACACCACGTCATTCTTCTC-5′ (SEQ ID NO: 18).

Luciferase reporter assay: Luciferase assays were performed with the dual-luciferase reporter assay kit (Promega). Briefly, cells were lysed in 1× passive lysis buffer (Promega) and 25 μL of lysate was used for assay with a tube luminometer using an integration time of 10 s. Transfection efficiency was normalized with the co-transfected pRL-SV40 Renilla luciferase expression plasmid [8, 11].

Immunoblotting: Immunoblotting was performed as described previously [11, 31]. Cell lysates for immunoblotting were prepared using RIPA buffer (Abcam, Waltham, MA) and included a protease and phosphatase inhibitor cocktail (Sigma, St. Louis, MO). Total protein extracts were obtained by centrifugation at 21,000×g for 30 min at 4° C. The clear supernatants were transferred to new tubes and quantified using the Bradford reagent with BSA as standard (BioRad, Hercules. CA). Equal amounts of cell lysates were separated by sodium dodecyl sulphate-polyacrylamide gel electrophoresis (SDS-PAGE), transferred to PVDF membranes (BioRad), and blocked with 5% non-fat dry milk. Incubation with primary antibodies (used at a concentration of 1:500) was performed overnight at 4° C. The PGC1α (catalog no. 2178), PINK1 (6946), LC3A/B (12741), phosphoserine-65 ubiquitin (62802), HIF2α (59973), TOM20 (72610), GAPDH (8884), actin (12748), and tubulin (9099) antibodies were obtained from Cell Signaling (Danvers, MA). The MNRR1 (19424-1), MTCO2 (55070-1), PHD1 (12984-1) and PHD3 (18325-1) antibodies were obtained from Proteintech (Chicago, IL). Incubation with secondary antibodies (1:5000) was performed for 2 h at room temperature. For detection after immunoblotting, the SuperSignal™ West Pico PLUS substrate (ThermoFisher, Waltham, MA) was used to generate chemiluminescence signal, which was detected with X-Ray film (RadTech, Vassar, MI).

Immunofluorescence: Cells plated on glass cover slips were fixed with 3.7% formaldehyde (prepared in 1× PBS) at room temperature for 15 min, followed by permeabilization with 0.15% Triton X-100 (prepared in distilled water) for 2 min, and then blocked with 5% bovine serum albumin (BSA) (prepared in 1× PBS, 0.1% TWEEN-20 (PBST)) for 1 h at room temperature. Cells were washed with PBST then incubated for 1 h at room temperature in primary antibody solution containing Coralite® 594 conjugated mouse monoclonal anti-CHCHD2 IgG (1:100, Proteintech, Cat. No. CL594-66302) prepared in PBST. Cells were washed 3 times with PBST for 5 min each and mounted with Vectashield vibrance with DAPI (Cat. #H-1800-10, Vector Labs, Newark, CA). Cells were imaged at 63× on the confocal 60 μm disk setting with the BioTek Cytation C10 using the Gen5 software (Agilent). Six fields for each group were taken and z-stacks of 25 slices (±3 slices) were performed for each field followed by a z-projection and image deconvolution. Corrected total fluorescence for each field to determine MNRR1 content was calculated using FIJI (National Institutes of Health).

MitochondrialDNA Levels: Total genomic DNA was isolated from cells expressing each of the mutants using the Invitrogen PureLink Genomic DNA Mini Kit (Thermo Fisher Scientific, K1820-01) and analyzed by real-time PCR as above. The primer sequences used to amplify mtDNA and GAPDH were as follows: mtDNA forward: 5′-CCTCCCTGTACGAAAGGAC-3′ (SEQ ID NO: 19); reverse: 5′-GCGATTAGAATGGGTACAATG-3′(SEQ ID NO: 20); GAPDH forward: 5′-GAGTCAACGGATTTGGTCGT-3′ (SEQ ID NO: 21); reverse: 5′-TTGATTTTGGAGGGATCTCG-3′ (SEQ ID NO: 22).

Restriction Enzyme Digestion: DNA was analyzed for the MELAS mutation as described previously [12]. The A→G mutation creates a new Haelll site at position 3243 that can be amplified by PCR using primers corresponding to the light-strand positions 3116 to 3134 and to the heavy-strand positions 3353 to 3333. Equal amounts of the resulting products were digested with the restriction enzyme Haelll (New England Biolabs) and electrophoresed on a 2.5% agarose gel.

ROS measurements: Total cellular ROS measurements were performed with CM-H2DCFDA (Life Technologies). Cells were distributed into 96-well plates at 2.5×10⁴ cells per well and incubated for 24 h or as described in specific experiments. Cells were then treated with 10 μM CM-H2DCFDA in serum- and antibiotic-free medium for 1 h. Cells were washed twice in phosphate buffered saline and analyzed for fluorescence on a BioTek Synergy H1 Microplate Reader (Agilent).

Intact cellular oxygen consumption and measurement of ATP: Cellular oxygen consumption was measured with a Seahorse XFe24 Bioanalyzer (Agilent). Cells were plated at a concentration of 3.5×10⁴ per well a day prior to treatment and basal oxygen consumption was measured 48 h after treatments, as described [8, 31]. For ATP levels, Agilent Seahorse ATP Real-Time rate assay kit was used per manufacturer's instructions.

Chromatin Immunoprecipitation-qPCR (ChIP-qPCR): Chromatin immunoprecipitation was performed per manufacturer's instructions using SimpleChlP® Enzymatic Chromatin IP Kit (Cell Signaling, 9002). Briefly, 2×10⁷ cells were fixed with formaldehyde to crosslink and chromatin was digested into ˜150-900 bp fragments using a combination of micrococcal nuclease and sonication. 2% of the sample was stored as input control. This digested chromatin was immunoprecipitated using the using the HIF2α antibody (Cell Signaling, 59973). Samples were eluted and the crosslinking was reversed. The eluted DNA and the input controls were purified and tested for relative amplification using qPCR analysis. The primers used were as follows: forward: 5′-ATCTTCCGGTCTCCTCAGAA-3′ (SEQ ID NO: 23); reverse: 3′-AAACCCTGCGATGGTCTCA-5′ (SEQ ID NO: 24).

Statistical Analysis: All statistical analyses were performed with the two-sided Wilcoxon rank sum test using MSTAT version 6.1.1 (N. Drinkwater, University of Wisconsin-Madison). *P<0.05; **P<0.005.

### REFERENCES

1. Nass, S. and M. M. Nass, Intramitochondrial Fibers with DNA Characteristics. Ii. Enzymatic and Other Hydrolytic Treatments. J Cell Biol, 1963. 19(3): p. 613-29.
2. Nass, M. M. and S. Nass, Intramitochondrial Fibers with DNA Characteristics. I. Fixation and Electron Staining Reactions. J Cell Biol, 1963. 19(3): p. 593-611.
3. Chalmers, R. M. and A. H. Schapira, Clinical, biochemical and molecular genetic features of Leber's hereditary optic neuropathy. Biochim Biophys Acta, 1999. 1410(2): p. 147-58.
4. Pitceathly, R. D. and R. McFarland, Mitochondrial myopathies in adults and children: management and therapy development. Curr Opin Neurol, 2014. 27(5): p. 576-82.
5. Schaefer, A. M., R. W. Taylor, D. M. Turnbull, and P. F. Chinnery, The epidemiology of mitochondrial disorders—past, present and future. Biochim Biophys Acta, 2004. 1659(2-3): p. 115-20.
6. Picard, M., J. Zhang, S. Hancock, O. Derbeneva, R. Golhar, P. Golik, S. O'Hearn, S. Levy, P. Potluri, M. Lvova, et al., Progressive increase in mtDNA 3243A>G heteroplasmy causes abrupt transcriptional reprogramming. Proc Natl Acad Sci USA, 2014. 111(38): p. E4033-42.
7. Baughman, J. M., R. Nilsson, V. M. Gohil, D. H. Arlow, Z. Gauhar, and V. K. Mootha, A computational screen for regulators of oxidative phosphorylation implicates SLIRP in mitochondrial RNA homeostasis. PLoS Genet, 2009. 5(8): p. e1000590.
8. Aras, S., H. Arrabi, N. Purandare, M. Huttemann, J. Kamholz, S. Zuchner, and L. I. Grossman, Abl2 kinase phosphorylates Bi-organellar regulator MNRR1 in mitochondria, stimulating respiration. Biochim Biophys Acta Mol Cell Res, 2017. 1864(2): p. 440-448.
9. Aras, S., M. Bai, I. Lee, R. Springett, M. Huttemann, and L. I. Grossman, MNRR1 (formerly CHCHD2) is a bi-organellar regulator of mitochondrial metabolism. Mitochondrion, 2015. 20: p. 43-51.
10. Liu, Y., H. V. Clegg, P. L. Leslie, J. Di, L. A. Tollini, Y. He, T. H. Kim, A. Jin, L. M. Graves, J. Zheng, et al., CHCHD2 inhibits apoptosis by interacting with Bcl-x L to regulate Bax activation. Cell Death Differ, 2015. 22(6): p. 1035-46.
11. Aras, S., O. Pak, N. Sommer, R. Finley, Jr., M. Huttemann, N. Weissmann, and L. I. Grossman, Oxygen-dependent expression of cytochrome c oxidase subunit 4-2 gene expression is mediated by transcription factors RBPJ, CXXC5 and CHCHD2. Nucleic Acids Res, 2013. 41(4): p.2255-66.
12. Aras, S., N. Purandare, S. Gladyck, M. Somayajulu-Nitu, K. Zhang, D. C. Wallace, and L. I. Grossman, Mitochondrial Nuclear Retrograde Regulator 1 (MNRR1) rescues the cellular phenotype of MELAS by inducing homeostatic mechanisms. Proc Natl Acad Sci USA, 2020. 117(50): p. 32056-32065.
13. Purandare, N., N. Gomez-Lopez, M. Arenas-Hernandez, J. Galaz, R. Romero, Y. Xi, A. M. Fribley, L. I. Grossman, and S. Aras, The MNRR1 activator nitazoxanide abrogates lipopolysaccharide-induced preterm birth in mice. Placenta, 2023. 140: p. 66-71.
14. Stockis, A., X. Deroubaix, R. Lins, B. Jeanbaptiste, P. Calderon, and J. F. Rossignol, Pharmacokinetics of nitazoxanide after single oral dose administration in 6 healthy volunteers. Int J Clin Pharmacol Ther, 1996. 34(8): p. 349-51.
15. Koyano, F., K. Okatsu, H. Kosako, Y. Tamura, E. Go, M. Kimura, Y. Kimura, H. Tsuchiya, H. Yoshihara, T. Hirokawa, et al., Ubiquitin is phosphorylated by PINK1 to activate parkin. Nature, 2014. 510(7503): p. 162-6.
16. Purandare, N., Y. Kunji, Y. Xi, R. Romero, N. Gomez-Lopez, A. Fribley, L. I. Grossman, and S. Aras, Lipopolysaccharide induces placental mitochondrial dysfunction in murine and human systems by reducing MNRR1 levels via a TLR4-independent pathway. iScience, 2022. 25(11): p. 105342.
17. Walker, M. A., N. Slate, A. Alejos, S. Volpi, R. S. lyengar, D. Sweetser, K. B. Sims, and J. E. Walter, Predisposition to infection and SIRS in mitochondrial disorders: 8 years' experience in an academic center. J Allergy Clin Immunol Pract, 2014. 2(4): p. 465-468, 468 el.
18. van Gisbergen, M. W., K. Offermans, A. M. Voets, N. G. Lieuwes, R. Biemans, R. F. Hoffmann,
L. J. Dubois, and P. Lambin, Mitochondrial Dysfunction Inhibits Hypoxia-Induced HIF-1alpha Stabilization and Expression of Its Downstream Targets. Front Oncol, 2020. 10: p. 770.
19. Diaz-Trelles, R., M. C. Scimia, P. Bushway, D. Tran, A. Monosov, E. Monosov, K. Peterson, S. Rentschler, P. Cabrales, P. Ruiz-Lozano, et al., Notch-independent RBPJ controls angiogenesis in the adult heart. Nat Commun, 2016. 7: p. 12088.
20. Xie, C., X. Gao, D. Sun, Y. Zhang, K. W. Krausz, X. Qin, and F. J. Gonzalez, Metabolic Profiling of the Novel Hypoxia-Inducible Factor 2alpha Inhibitor PT2385 In Vivo and In Vitro. Drug Metab Dispos, 2018. 46(4): p. 336-345.
21. Fujita, N., D. Markova, D. G. Anderson, K. Chiba, Y. Toyama, I. M. Shapiro, and M. V. Risbud, Expression of prolyl hydroxylases (PHDs) is selectively controlled by HIF-1 and HIF-2 proteins in nucleus pulposus cells of the intervertebral disc: distinct roles of PHD2 and PHD3 proteins in controlling HIF-1alpha activity in hypoxia. J Biol Chem, 2012. 287(20): p. 16975-86.
22. Appelhoff, R. J., Y. M. Tian, R. R. Raval, H. Turley, A. L. Harris, C. W. Pugh, P. J. Ratcliffe, and J. M. Gleadle, Differential function of the prolyl hydroxylases PHD1, PHD2, and PHD3 in the regulation of hypoxia-inducible factor. J Biol Chem, 2004. 279(37): p. 38458-65.
23. Miikkulainen, P., H. Hogel, F. Seyednasrollah, K. Rantanen, L. L. Elo, and P. M. Jaakkola, Hypoxia-inducible factor (HIF)-prolyl hydroxylase 3 (PHD3) maintains high HIF2A mRNA levels in clear cell renal cell carcinoma. J Biol Chem, 2019. 294(10): p. 3760-3771.
24. Taniguchi, C. M., E. C. Finger, A. J. Krieg, C. Wu, A. N. Diep, E. L. LaGory, K. Wei, L. M. McGinnis,
J. Yuan, C. J. Kuo, et al., Cross-talk between hypoxia and insulin signaling through Phd3 regulates hepatic glucose and lipid metabolism and ameliorates diabetes. Nat Med, 2013. 19(10): p. 1325-30.
25. Li, X. W., R. Z. He, Y. Li, and Z. F. Ruan, Tizoxanide mitigates inflammatory response in LPS-induced neuroinflammation in microglia via restraining p38/MAPK pathway. Eur Rev Med Pharmacol Sci, 2020. 24(11): p. 6446-6454.
26. Lokhande, A. S. and P. V. Devarajan, A review on possible mechanistic insights of Nitazoxanide for repurposing in COVID-19. Eur J Pharmacol, 2021. 891: p. 173748.
27. He, X., W. Hu, F. Meng, and X. Li, Design, Synthesis, and Pharmacokinetic Evaluation of O-Carbamoyl Tizoxanide Prodrugs. Med Chem, 2022. 18(1): p. 140-150.
28. Neary, M., U. Arshad, L. Tatham, H. Pertinez, H. Box, R. K. R. Rajoli, A. Valentijn, J. Sharp, S. P. Rannard, G. A. Biagini, et al., Quantitation of tizoxanide in multiple matrices to support cell culture, animal and human research. J Chromatogr B Analyt Technol Biomed Life Sci, 2023. 1228: p. 123823.
29. Guo, S., F. Li, B. Wang, Y. Zhao, X. Wang, H. Wei, K. Yu, and X. Hai, Analysis of tizoxanide, active metabolite of nitazoxanide, in rat brain tissue and plasma by UHPLC-MS/MS. Biomed Chromatogr, 2020. 34(2): p. e4716.
30. Birmingham, A., L. M. Selfors, T. Forster, D. Wrobel, C. J. Kennedy, E. Shanks, J. Santoyo-Lopez, D. J. Dunican, A. Long, D. Kelleher, et al., Statistical methods for analysis of high-throughput RNA interference screens. Nat Methods, 2009. 6(8): p. 569-75.
31. Purandare, N., M. Somayajulu, M. Huttemann, L. I. Grossman, and S. Aras, The cellular stress proteins CHCHD10 and MNRR1 (CHCHD2): Partners in mitochondrial and nuclear function and dysfunction. J Biol Chem, 2018. 293(17): p. 6517-6529.
32. Zhang, L., S. Peng, X. Dai, W. Gan, X. Nie, W. Wei, G. Hu, and J. Guo, Tumor suppressor SPOP ubiquitinates and degrades EgIN2 to compromise growth of prostate cancer cells. Cancer Lett, 2017. 390: p. 11-20.
33. Wang, Y., X. Li, W. Liu, B. Li, D. Chen, F. Hu, L. Wang, X. M. Liu, R. Cui, and R. Liu, MicroRNA-1205, encoded on chromosome 8q24, targets EGLN3 to induce cell growth and contributes to risk of castration-resistant prostate cancer. Oncogene, 2019. 38(24): p. 4820-4834.
34. Aras, S., H. Arrabi, N. Purandare, M. Huttemann, J. Kamholz, S. Zuchner, and L. I. Grossman, Abl2 kinase phosphorylates Bi-organellar regulator MNRR1 in mitochondria, stimulating respiration. Biochim Biophys Acta Mol Cell Res, 2017. 1864(2): p. 440-448.
35. Aras, S., M. Bai, I. Lee, R. Springett, M. Huttemann, and L. I. Grossman, MNRR1 (formerly CHCHD2) is a bi-organellar regulator of mitochondrial metabolism. Mitochondrion, 2015. 20: p. 43-51.
36. Liu, Y., H. V. Clegg, P. L. Leslie, J. Di, L. A. Tollini, Y. He, T. H. Kim, A. Jin, L. M. Graves, J. Zheng, et al., CHCHD2 inhibits apoptosis by interacting with Bcl-x L to regulate Bax activation. Cell Death Differ, 2015. 22(6): p. 1035-46.
37. Aras, S., O. Pak, N. Sommer, R. Finley, Jr., M. Huttemann, N. Weissmann, and L. I. Grossman, Oxygen-dependent expression of cytochrome c oxidase subunit 4-2 gene expression is mediated by transcription factors RBPJ, CXXC5 and CHCHD2. Nucleic Acids Res, 2013. 41(4): p.2255-66.
38. Aras, S., N. Purandare, S. Gladyck, M. Somayajulu-Nitu, K. Zhang, D. C. Wallace, and L. I. Grossman, Mitochondrial Nuclear Retrograde Regulator 1 (MNRR1) rescues the cellular phenotype of MELAS by inducing homeostatic mechanisms. Proc Natl Acad Sci USA, 2020. 117(50): p. 32056-32065.
39. Purandare, N., N. Gomez-Lopez, M. Arenas-Hernandez, J. Galaz, R. Romero, Y. Xi, A. M. Fribley, L. I. Grossman, and S. Aras, The MNRR1 activator nitazoxanide abrogates lipopolysaccharide-induced preterm birth in mice. Placenta, 2023. 140: p. 66-71.
40. Stockis, A., X. Deroubaix, R. Lins, B. Jeanbaptiste, P. Calderon, and J. F. Rossignol, Pharmacokinetics of nitazoxanide after single oral dose administration in 6 healthy volunteers. Int J Clin Pharmacol Ther, 1996. 34(8): p. 349-51.
41. Koyano, F., K. Okatsu, H. Kosako, Y. Tamura, E. Go, M. Kimura, Y. Kimura, H. Tsuchiya, H. Yoshihara, T. Hirokawa, et al., Ubiquitin is phosphorylated by PINK1 to activate parkin. Nature, 2014. 510(7503): p. 162-6.
42. Purandare, N., Y. Kunji, Y. Xi, R. Romero, N. Gomez-Lopez, A. Fribley, L. I. Grossman, and S. Aras, Lipopolysaccharide induces placental mitochondrial dysfunction in murine and human systems by reducing MNRR1 levels via a TLR4-independent pathway. iScience, 2022. 25(11): p. 105342.
43. Walker, M. A., N. Slate, A. Alejos, S. Volpi, R. S. lyengar, D. Sweetser, K. B. Sims, and J. E. Walter, Predisposition to infection and SIRS in mitochondrial disorders: 8 years' experience in an academic center. J Allergy Clin Immunol Pract, 2014. 2(4): p. 465-468, 468 el.
44. van Gisbergen, M. W., K. Offermans, A. M. Voets, N. G. Lieuwes, R. Biemans, R. F. Hoffmann, L. J. Dubois, and P. Lambin, Mitochondrial Dysfunction Inhibits Hypoxia-Induced HIF-1alpha Stabilization and Expression of Its Downstream Targets. Front Oncol, 2020. 10: p. 770.
45. Diaz-Trelles, R., M. C. Scimia, P. Bushway, D. Tran, A. Monosov, E. Monosov, K. Peterson, S. Rentschler, P. Cabrales, P. Ruiz-Lozano, et al., Notch-independent RBPJ controls angiogenesis in the adult heart. Nat Commun, 2016. 7: p. 12088.
46. Xie, C., X. Gao, D. Sun, Y. Zhang, K. W. Krausz, X. Qin, and F. J. Gonzalez, Metabolic Profiling of the Novel Hypoxia-Inducible Factor 2alpha Inhibitor PT2385 In Vivo and In Vitro. Drug Metab Dispos, 2018. 46(4): p. 336-345.
47. Fujita, N., D. Markova, D. G. Anderson, K. Chiba, Y. Toyama, I. M. Shapiro, and M. V. Risbud, Expression of prolyl hydroxylases (PHDs) is selectively controlled by HIF-1 and HIF-2 proteins in nucleus pulposus cells of the intervertebral disc: distinct roles of PHD2 and PHD3 proteins in controlling HIF-1alpha activity in hypoxia. J Biol Chem, 2012. 287(20): p. 16975-86.
48. Appelhoff, R. J., Y. M. Tian, R. R. Raval, H. Turley, A. L. Harris, C. W. Pugh, P. J. Ratcliffe, and J. M. Gleadle, Differential function of the prolyl hydroxylases PHD1, PHD2, and PHD3 in the regulation of hypoxia-inducible factor. J Biol Chem, 2004. 279(37): p. 38458-65.
49. Miikkulainen, P., H. Hogel, F. Seyednasrollah, K. Rantanen, L. L. Elo, and P. M. Jaakkola, Hypoxia-inducible factor (HIF)-prolyl hydroxylase 3 (PHD3) maintains high HIF2A mRNA levels in clear cell renal cell carcinoma. J Biol Chem, 2019. 294(10): p. 3760-3771.
50. Taniguchi, C. M., E. C. Finger, A. J. Krieg, C. Wu, A. N. Diep, E. L. LaGory, K. Wei, L. M. McGinnis,
J. Yuan, C. J. Kuo, et al., Cross-talk between hypoxia and insulin signaling through Phd3 regulates hepatic glucose and lipid metabolism and ameliorates diabetes. Nat Med, 2013. 19(10): p. 1325-30.
51. Li, X. W., R. Z. He, Y. Li, and Z. F. Ruan, Tizoxanide mitigates inflammatory response in LPS-induced neuroinflammation in microglia via restraining p38/MAPK pathway. Eur Rev Med Pharmacol Sci, 2020. 24(11): p. 6446-6454.
52. Lokhande, A. S. and P. V. Devarajan, A review on possible mechanistic insights of Nitazoxanide for repurposing in COVID-19. Eur J Pharmacol, 2021. 891: p. 173748.
53. He, X., W. Hu, F. Meng, and X. Li, Design, Synthesis, and Pharmacokinetic Evaluation of O-Carbamoyl Tizoxanide Prodrugs. Med Chem, 2022. 18(1): p. 140-150.
54. Neary, M., U. Arshad, L. Tatham, H. Pertinez, H. Box, R. K. R. Rajoli, A. Valentijn, J. Sharp, S. P. Rannard, G. A. Biagini, et al., Quantitation of tizoxanide in multiple matrices to support cell culture, animal and human research. J Chromatogr B Analyt Technol Biomed Life Sci, 2023. 1228: p. 123823.
55. Guo, S., F. Li, B. Wang, Y. Zhao, X. Wang, H. Wei, K. Yu, and X. Hai, Analysis of tizoxanide, active metabolite of nitazoxanide, in rat brain tissue and plasma by UHPLC-MS/MS. Biomed Chromatogr, 2020. 34(2): p. e4716.
56. Birmingham, A., L. M. Selfors, T. Forster, D. Wrobel, C. J. Kennedy, E. Shanks, J. Santoyo-Lopez, D. J. Dunican, A. Long, D. Kelleher, et al., Statistical methods for analysis of high-throughput RNA interference screens. Nat Methods, 2009. 6(8): p. 569-75.
57. Purandare, N., M. Somayajulu, M. Huttemann, L. I. Grossman, and S. Aras, The cellular stress proteins CHCHD10 and MNRR1 (CHCHD2): Partners in mitochondrial and nuclear function and dysfunction. J Biol Chem, 2018. 293(17): p. 6517-6529.
58. Zhang, L., S. Peng, X. Dai, W. Gan, X. Nie, W. Wei, G. Hu, and J. Guo, Tumor suppressor SPOP ubiquitinates and degrades EgIN2 to compromise growth of prostate cancer cells. Cancer Lett, 2017. 390: p. 11-20.
59. Wang, Y., X. Li, W. Liu, B. Li, D. Chen, F. Hu, L. Wang, X. M. Liu, R. Cui, and R. Liu, MicroRNA-1205, encoded on chromosome 8q24, targets EGLN3 to induce cell growth and contributes to risk of castration-resistant prostate cancer. Oncogene, 2019. 38(24): p. 4820-4834.

### VII. Closing Paragraphs

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; +19% of the stated value; ±18% of the stated value; +17% of the stated value; +16% of the stated value; ±15% of the stated value; +14% of the stated value; ±13% of the stated value; +12% of the stated value; +11% of the stated value; +10% of the stated value; ±9% of the stated value; ±8% of the stated value; +7% of the stated value; ±6% of the stated value; ±5% of the stated value; +4% of the stated value; ±3% of the stated value; +2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds, nucleic acid, and amino acids sequences referenced herein that are available in a public database, the information in the database entry is incorporated herein by reference as of the date of an application in the priority chain in which the database identifier for that compound or sequence was first included in the text.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8^{th} Edition (Ed. J. Law & R. Rennie, Oxford University Press, 2020).

What is claimed is:

1. A method of delaying inflammation-induced labor in a pregnant subject, comprising:
selecting a pregnant subject; and
activating MNRR1 in a cell of the pregnant subject.

2. The method of claim 1, wherein the cell is a placental cell.

3. The method of claim 1, wherein activating MNRR1 in the cell of the pregnant subject comprises administering an effective amount of a MNRR1 activating agent to the pregnant subject.

4. The method of claim 3, wherein the MNRR1 activating agent comprises at least one active tizoxanide moiety.

5. The method of claim 3, wherein the MNRR1 activating agent comprises nitazoxanide.

6. The method of claim 3, wherein the MNRR1 activating agent is administered prior to exposure of the pregnant subject to an inflammation-causing agent.

7. The method of claim 6, wherein the inflammation-causing agent comprises a microbe.

8. The method of claim 3, wherein the administering is oral administration.

9. The method of claim 1, wherein delaying inflammation-induced labor in the pregnant subject prevents premature birth.

10. The method of claim 1, comprising administering an effective amount of progesterone to the pregnant subject.

11. The method of claim 5, wherein the nitazoxanide is administered to the pregnant subject in a dose of 500 mg twice daily.

12. A method of reducing or preventing pre-term labor in a pregnant subject, comprising:
selecting a pregnant subject; and
administering a MNRR1 activating agent to the pregnant subject.

13. The method of claim 12, wherein the MNRR1 activating agent comprises at least one active tizoxanide moiety.

14. The method of claim 12, wherein the MNRR1 activating agent comprises nitazoxanide, optionally wherein the nitazoxanide is administered to the pregnant subject in a dose of 500 mg twice daily.

15. The method of claim 12, wherein the MNRR1 activating agent is administered prior to exposure of the pregnant subject to an inflammation-causing agent.

16. The method of claim 15, wherein the inflammation-causing agent comprises a microbe.

17. The method of claim 12, wherein the administering is oral administration.

18. The method of claim 12, wherein reducing or preventing pre-term labor in the pregnant subject prevents premature birth.

19. The method of claim 12, comprising administering an effective amount of progesterone to the pregnant subject.

20. A method of treating an inflammatory disorder or a mitochondria-associated disorder in a subject, comprising activating Mitochondrial Nuclear Retrograde, Regulator 1 (MNRR1) in a cell of the subject.


SEQUENCE LISTING (27 sequences)

SEQ ID NO: 1 - DNA, 18 bp, Synthetic construct
  source 1..18: mol_type = other DNA
atggcccagatggctacc

SEQ ID NO: 2 - DNA, 20 bp, Synthetic construct
  source 1..20: mol_type = other DNA
acctcacacgagtcttggtc

SEQ ID NO: 3 - DNA, 20 bp, Synthetic construct
  source 1..20: mol_type = other DNA
tcctccctggagaagagcta

SEQ ID NO: 4 - DNA, 20 bp, Synthetic construct
  source 1..20: mol_type = other DNA
tcacactgcaactgtaggca

SEQ ID NO: 5 - DNA, 20 bp, Synthetic construct
  source 1..20: mol_type = other DNA
tgtagcaaaccctcaagctg

SEQ ID NO: 6 - DNA, 20 bp, Synthetic construct
  source 1..20: mol_type = other DNA
tggtctggttccagttggag

SEQ ID NO: 7 - DNA, 20 bp, Synthetic construct
  source 1..20: mol_type = other DNA
cattaaggagaagctgtgct

SEQ ID NO: 8 - DNA, 20 bp, Synthetic construct
  source 1..20: mol_type = other DNA
aggtgctttgatggaagttg

SEQ ID NO: 9 - DNA, 23 bp, Synthetic construct
  source 1..23: mol_type = other DNA
cacacatgggtcacgccattact

SEQ ID NO: 10 - DNA, 24 bp, Synthetic construct
  source 1..24: mol_type = other DNA
ttctgggcacactccagaaactgt

SEQ ID NO: 11 - DNA, 20 bp, Synthetic construct
  source 1..20: mol_type = other DNA
ccagtaagtgcgggtcataa

SEQ ID NO: 12 - DNA, 20 bp, Synthetic construct
  source 1..20: mol_type = other DNA
ggcctcactaaaccatccaa

SEQ ID NO: 13 - DNA, 20 bp, Synthetic construct
  source 1..20: mol_type = other DNA
acatcgagccactctttgac

SEQ ID NO: 14 - DNA, 19 bp, Synthetic construct
  source 1..19: mol_type = other DNA
ccataaaactacggttcct

SEQ ID NO: 15 - DNA, 21 bp, Synthetic construct
  source 1..21: mol_type = other DNA
tcaaggagaggtctaaggcaa

SEQ ID NO: 16 - DNA, 18 bp, Synthetic construct
  source 1..18: mol_type = other DNA
agcgacgtagtggacgta

SEQ ID NO: 17 - DNA, 20 bp, Synthetic construct
  source 1..20: mol_type = other DNA
caccaagggtcaggtagtaa

SEQ ID NO: 18 - DNA, 20 bp, Synthetic construct
  source 1..20: mol_type = other DNA
ctcttcttactgcaccacaa

SEQ ID NO: 19 - DNA, 19 bp, Synthetic construct
  source 1..19: mol_type = other DNA
cctccctgtacgaaaggac

SEQ ID NO: 20 - DNA, 21 bp, Synthetic construct
  source 1..21: mol_type = other DNA
gcgattagaatgggtacaatg

SEQ ID NO: 21 - DNA, 20 bp, Synthetic construct
  source 1..20: mol_type = other DNA
gagtcaacggatttggtcgt

SEQ ID NO: 22 - DNA, 20 bp, Synthetic construct
  source 1..20: mol_type = other DNA
ttgattttggagggatctcg

SEQ ID NO: 23 - DNA, 20 bp, Synthetic construct
  source 1..20: mol_type = other DNA
atcttccggtctcctcagaa

SEQ ID NO: 24 - DNA, 19 bp, Synthetic construct
  source 1..19: mol_type = other DNA
actctggtagcgtcccaaa

SEQ ID NO: 25 - DNA, 152 bp, Synthetic construct
  source 1..152: mol_type = other DNA
cccatcttccggtctcctcagaagtcgcttagctcttcggtggttgtcccacgtccggaggcctagccgtcgcttacctaggatgccgcgtggaagccgaagccgcacctcccgcatggcccctccggccaggtgagaccatcgcagggttt

SEQ ID NO: 26 - DNA, 12 bp, Synthetic construct
  source 1..12: mol_type = other DNA
tcccacgtccgg

SEQ ID NO: 27 - DNA, 12 bp, Synthetic construct
  source 1..12: mol_type = other DNA
ccggacgtggga